(19) United States
(12) Patent Application Publication
(10) Pub. No.: US 2020/0190475 A1
(43) Pub. Date: Jun. 18, 2020

(54) METHODS FOR IDENTIFYING AND ISOLATING CARDIAC STEM CELLS AND METHODS FOR MAKING AND USING THEM

(71) Applicant: San Diego State University (SDSU) Foundation, dba San Diego State University Research Foundation, San Diego, CA (US)

(72) Inventor: Mark Alan SUSSMAN, San Diego, CA (US)

(21) Appl. No.: 16/488,464

(22) PCT Filed: Feb. 23, 2018
(86) PCT No.: PCT/US2018/019435

§ 371 Date: Aug. 23, 2019

Related U.S. Application Data
(60) Provisional application No. 62/462,902, filed on Feb. 23, 2017.

(51) Int. Cl. C12N 5/077; A61K 35/34; C12Q 1/6841; C12Q 1/686; C12Q 1/6851

(52) U.S. Cl. CPC C12N 5/0657; A61K 35/34; C12Q 1/6841; C12Q 2600/158; C12Q 1/6851; C12Q 2561/113; C12Q 1/686

(57) ABSTRACT

Provided are methods for identifying and isolating cardiac stem or cardiac progenitor cells, and methods for making and using them, including comprising identifying cells expressing the Lin28 polypeptide, cells that are Lin28+; cells that are Lin28+ can be isolated using polypeptides or antibodies that can specifically or non-specifically bind Lin28. Provided are methods for inducing or accelerating cardiogenesis in the mammalian heart, or for treating a heart genetic defect, injury or dysfunction, or an injury or dysfunction subsequent to an ischemic injury or a heart failure, or an injury or dysfunction resulting from a myocardial infarction, comprising administering to an individual one or more of the Lin28+ cardiac stem cells or cardiac progenitor cells, including Lin28+ cardiac stem cells or cardiac progenitor cells isolated by a method as provided herein, or a Lin28+ interstitial population provided herein, or administrating to a patient a product of manufacture provided herein.

### RELATED APPLICATIONS

This Patent Convention Treaty (PCT) International Application claims the benefit of priority to U.S. Provisional Application No. 62/462,902, filed Feb. 23, 2017. The aforementioned application is expressly incorporated herein by reference in its entirety and for all purposes.

### STATEMENT AS TO FEDERALLY SPONSORED RESEARCH

This invention was made with government support under grant numbers R01HL067245, R37HL091102, R01HL105759, R01HL113647, R01HL117163, P01HL085577, and R01HL122525, National Institutes of Health (NIH), DHHS. The government has certain rights in the invention.

### TECHNICAL FIELD

This invention generally relates to cell and molecular and stem cell biology and regenerative medicine. In alternative embodiments, provided are methods for identifying and isolating cardiac stem cells or cardiac progenitor cells, and methods for making and using them. In alternative embodiments, provided are methods for identifying and isolating cardiac stem cells or cardiac progenitor cells comprising identifying cells expressing the Lin28 polypeptide (or Lin-28 homolog A, which is a protein that in humans is encoded by the LIN28 gene), i.e., cells that are Lin28+. In alternative embodiments, cells that are Lin28+ are isolated using polypeptides or other compositions, e.g., antibodies, that can specifically or non-specifically bind Lin28. In alternative embodiments, provided are methods for inducing or accelerating cardiogenesis in the mammalian (e.g., human) heart, or for treating, preventing, reversing the effects of or ameliorating a heart genetic defect, injury or dysfunction, or an injury or dysfunction subsequent to (or following, or immediately after) an ischemic injury or a heart failure, or an injury or dysfunction resulting from a myocardial infarction (MI), comprising administering to an individual in need thereof one or more of the Lin28+ cardiac stem cells or cardiac progenitor cells provided herein, including Lin28+ cardiac stem cells or cardiac progenitor cells isolated by a method provided herein, or a Lin28+ Interstitial Population (LIP) as provided herein, or administrating to a patient a product of manufacture as provided herein.

### BACKGROUND

Cellular therapy using stem cells derived from the bone marrow and cells of cardiac origin are validated to treat damage after myocardial infarction (MI) in both small animal models and human clinical trials. Application of cellular therapy of MI is hindered by results of little to no improvement in cardiac function after long-term follow up studies using a variety of stem cell strategies. The inherent limitation of autologous stem cell therapy is that cells derived from aged organs have increased expression of senescent markers and acquisition of chromosomal abnormalities leading to undesirable cellular characteristics such as slowed proliferation and increased susceptibility to cellular death. Furthermore, based on animal models, cellular survival and engraftment is hindered by adverse inflammation, inhibiting the ability of transplanted stem cells to efficiently differentiate into cardiac cells. Improvement of stem cell engraftment and survival has been attempted by co-injection of stem cells with biomaterials, cytokines and growth factors, or genetically enhancing cells with pro-survival and anti-apoptotic genes.

The heart is capable of limited regeneration, as evidenced by cardiomyocyte re-entry into the cell cycle and production of new mono-nucleated myocytes during aging and after pathological damage.

The regenerative potential of stem cells in a clinical setting is still largely unrealized, as stem cells are suggested to function through a variety of mechanisms for myocardial repair yet stem cells are inherently limited because of origin and potency status. Progress is hampered by limited options for cardiac-associated stem cell markers used to enrich for cells with therapeutic potential. There remains a need for better identification and isolation of cardiac stem cells.

Lin-28 homolog A is a protein that in humans is encoded by the LIN28 gene. LIN28 encodes an RNA-binding protein that binds to and enhances the translation of the IGF-2 (insulin-like growth factor 2) mRNA. LIN28 is thought to regulate the self-renewal of stem cells, and LIN28 is highly expressed in human embryonic stem cells and can enhance the efficiency of the formation of induced pluripotent stem (iPS) cells from human fibroblasts.

### SUMMARY

In alternative embodiments, provided are methods for identifying or isolating a cardiac stem cell, an adult cardiac stem cell or a cardiac progenitor cell, comprising: identifying a cell or cells expressing: (i) a Lin28 polypeptide, or a Lin-28 homolog A, a protein that in humans is encoded by the LIN28 gene, or a Lin28+ cell, or (ii) a transcript (mRNA) encoding the Lin28 polypeptide;

wherein the method optionally further comprises a step (b) comprising isolating the identified Lin28+ cells,

and optionally cells that are Lin28+ are isolated using polypeptides or compositions that can specifically or non-specifically bind Lin28, or substantially bind Lin28, and optionally the polypeptides are Lin28-binding antibodies or Lin28-binding fragments thereof,

and optionally cells expressing the transcripts (mRNA) encoding the Lin28 polypeptide are identified using fluorescence in situ hybridization (FISH) or polymerase chain reaction (PCR), and optionally the PCR comprises a real-time PCR (also known as quantitative polymerase chain reaction (qPCR)), or in situ hybridization (ISH),

and optionally the cells are human or non-human (animal) cells.

In alternative embodiments, provided are macrocellular structures, a cardiocluster of cells, or an artificially configured plurality of cells, comprising:
(a) (i) a core region or cluster comprising a plurality of Lin28+ cardiac stem cells or Lin28+ cardiac progenitor cells, cells isolated using a method as provided herein, or a combination thereof; and
(ii) a second region or a peripheral region positioned: at least partially surrounding the outer surface of the core region or cluster, or at least partially around the core region or cluster, comprising a plurality of second cardiac stem cells, wherein the first cardiac stem cells and the second cardiac stem cells are different, and the second cardiac progenitor cells are selected from the group consisting of:
a plurality of mesenchymal stem cells or mesenchymal progenitor cells,
a plurality of endothelial progenitor cells or endothelial stem cells, and
a combination thereof; or
(b) (i) a core region or cluster comprising: a plurality of first cardiac stem cells and second cardiac stem cells, wherein the first cardiac stem cells and the second cardiac stem cells are different, and the first cardiac progenitor cells are selected from the group consisting of:
a plurality of mesenchymal stem cells or mesenchymal progenitor cells,
a plurality of endothelial progenitor cells or endothelial stem cells, and
a combination thereof; and

(ii) a second region or a peripheral region positioned: at least partially surrounding the outer surface of the core region or cluster, or at least partially around the core region or cluster, comprising a plurality of second cardiac stem cells comprising: a plurality of Lin28+ cardiac stem cells or Lin28+ cardiac progenitor cells, cells isolated using a method as provided herein, or a combination thereof.

In alternative embodiments, provided are macrocellular structures, cardiocluster of cells, or the artificially configured plurality of cells comprising a first, a second and a third plurality of stem cells or progenitor cells, wherein the first, second and third cardiac stem cells or progenitor cells are different cell types or comprises a different set of cells (wherein optionally the difference in the cell types is defined or determined by the set of cells having different genotypes or phenotypes),

and at least one of the first, second and third cardiac stem cells or progenitor cells comprise:

(i) a plurality of cardiac progenitor cells or cardiac stem cells,
a plurality of mesenchymal stem cells or mesenchymal progenitor cells,
a plurality of endothelial progenitor cells or endothelial stem cells, or
a combination thereof, and

(ii) a plurality of Lin28+ cardiac stem cells or Lin28+ cardiac progenitor cells, or cells isolated using a method as provided herein,

and optionally the plurality of third stem cell are positioned or configured: at least partially surrounding the outer surface of the second region or the core region or cluster, or at least partially around the second region or the core region or cluster,

and optionally the plurality of third stem cells or progenitor cells are positioned or configured in the core region or cluster,

and optionally the plurality of third stem cell are positioned or configured: at least partially surrounding the outer surface of the second region or peripheral region or the core region or cluster, or at least partially around the second region or peripheral region or the core region or cluster, and are also positioned or configured in the core region or cluster,

and optionally the plurality of third stem cells or progenitor cells are of non-cardiac origin, and optionally the third stem cells are mesenchymal stem cells of non-cardiac origin.

In alternative embodiments, the first, second, and third stem cells are of human origin or are of non-human (animal) origin, or, the core region or cluster, or the second or third region or peripheral region, comprises cells selected from the group consisting of: c-kit+ cardiac progenitor cells (CPCs), CD90+/CD105+ mesenchymal stem cells (MSCs) CD133+ endothelial progenitor cells (EPCs), and a combination thereof.

In alternative embodiments, provided are products of manufacture comprising: a cell or a plurality of Lin28+ cardiac stem cells or Lin28+ cardiac progenitor cells, or a cell or a plurality of cells isolated using a method as provided herein, or a macrocellular structure, a cardiocluster of cells or an artificially configured plurality of cells as provided herein,

wherein optionally the product of manufacture comprises drug delivery device, an implant, a catheter, a cartridge, an ampoule, a stent, or a medical device.

In alternative embodiments, provided are methods for:
inducing or accelerating cardiogenesis in the mammalian heart,
initiating, inducing or accelerating tissue repair or tissue regeneration, optionally a cardiac or heart tissue repair or heart tissue regeneration,
initiating, inducing or accelerating a cardiac muscle repair or tissue regeneration, a cardiac vasculature repair or tissue regeneration or a cardiac connective tissue repair or tissue regeneration,

comprising:

(a) providing:
a cell or a plurality of Lin28+ cardiac stem cells, or a cell or a plurality of Lin28+ cardiac progenitor cells,
a cell or plurality of cells isolated using a method as provided herein,
a Lin28+ interstitial population (LIP),
a macrocellular structure, a cardiocluster of cells, or an artificially configured plurality of cells as provided herein, or
a product of manufacture as provided herein; and

(b) introducing into, onto or approximate to (close to) the mammalian heart, or cardiac or heart tissue, or heart muscle, or cardiac vasculature or connective tissue: the plurality of Lin28+ cardiac stem cells or Lin28+ cardiac progenitor cells, or the plurality of cells isolated using a method as provided herein, or the Lin28+ interstitial population (LIP), or the macrocellular structure, the cardiocluster of cells or the artificially configured plurality of cells of step (a), or a product of manufacture as provided herein,

thereby inducing or accelerating cardiogenesis in the mammalian heart, or for repairing or regenerating the tissue, or the cardiac tissue, or the cardiac muscle, cardiac vasculature or cardiac connective tissue.

In alternative embodiments, the heart has an injury or dysfunction and the method is effective to treat the injury or dysfunction. In alternative embodiments, the injury or dysfunction: is an ischemic injury or a heart failure, or results from myocardial infarction (MI).

In alternative embodiments, provided are methods for treating or ameliorating a heart injury, an injury subsequent to (or following, or optionally from 1 minute to 12 hours after, or immediately after) a myocardial infarction (MI), a congenital or genetic heart defect, or a heart dysfunction, comprising:

(a) providing:
a cell or a plurality of Lin28+ cardiac stem cells, or a cell or a plurality of Lin28+ cardiac progenitor cells,
a cell or plurality of cells isolated using a method as provided herein,
a Lin28+ interstitial population (LIP),
a macrocellular structure, a cardiocluster of cells, or an artificially configured plurality of cells as provided herein, or
a product of manufacture as provided herein; and

(b) introducing into, onto or approximate (close to) to a mammalian heart, or administering to or applying to an individual in need thereof, the plurality of Lin28+ cardiac stem cells or Lin28+ cardiac progenitor cells, or the plurality of cells isolated using a method as provided herein, or the Lin28+ interstitial population (LIP), or the macrocellular structure, the cardiocluster of cells or the artificially configured plurality of cells as provided herein, or the a product of manufacture as provided herein,

thereby treating or ameliorating the heart injury, injury subsequent to (or following, or optionally from 1 minute to 12 hours after, or immediately after) a myocardial infarction (MI), congenital or genetic heart defect, or heart dysfunction.

In alternative embodiments, provided are methods for treating or ameliorating a heart injury, an injury subsequent to (or following, or optionally from 1 minute to 12 hours after, or immediately after) a myocardial infarction (MI), a congenital or genetic heart defect, or a heart dysfunction, comprising:

(a) providing: a product of manufacture as provided herein; and

(b) introducing into, onto or approximate to (close to) a mammalian heart, or administering to or applying to an individual in need thereof, the product of manufacture of (a),

thereby treating or ameliorating the heart injury, injury subsequent to (or following, or optionally from 1 minute to 12 hours after, or immediately after) a myocardial infarction (MI), the congenital or genetic heart defect, or the heart dysfunction.

In alternative embodiments, provided are Uses of:
a cell or a plurality of Lin28+ cardiac stem cells, or a cell or a plurality of Lin28+ cardiac progenitor cells,
a cell or plurality of cells isolated using a method as provided herein,
a Lin28+ interstitial population (LIP),
a macrocellular structure, a cardiocluster of cells, or an artificially configured plurality of cells as provided herein,
or a product of manufacture as provided herein, for, or in the manufacture of a medicament for:
   inducing or accelerating cardiogenesis in the mammalian heart,
   initiating, inducing or accelerating tissue repair or tissue regeneration, optionally a cardiac or heart tissue repair or heart tissue regeneration,
   initiating, inducing or accelerating a cardiac muscle repair or tissue regeneration, a cardiac vasculature repair or tissue regeneration or a cardiac connective tissue repair or tissue regeneration, or
   for treating or ameliorating a heart injury, an injury subsequent to (or following, or optionally from 1 minute to 12 hours after, or immediately after) a myocardial infarction (MI), a congenital or genetic heart defect, or a heart dysfunction,

wherein optionally the cell or plurality of cells, macrocellular structure or cardiocluster, or product of manufacture, are introduced into, onto or approximate to (close to) a mammalian heart, or are administered to or applied to an individual in need thereof.

In alternative embodiments, provided are: a cell or a plurality of Lin28+ cardiac stem cells, or a cell or a plurality of Lin28+ cardiac progenitor cells; a cell or plurality of cells isolated using a method as provided herein; a Lin28+ interstitial population (LIP); a macrocellular structure, a cardiocluster of cells, or an artificially configured plurality of cells as provided herein; or a product of manufacture as provided herein,

for, or for use in the manufacture of a medicament for:
inducing or accelerating cardiogenesis in the mammalian heart,
tissue repair or tissue regeneration, optionally a cardiac or heart tissue repair or heart tissue regeneration,
a cardiac muscle repair or tissue regeneration, a cardiac vasculature repair or tissue regeneration or a cardiac connective tissue repair or tissue regeneration, or
for treating or ameliorating a heart injury, an injury subsequent to (or following, or optionally from 1 minute to 12 hours after, or immediately after) a myocardial infarction (MI), a congenital or genetic heart defect, or a heart dysfunction,

wherein optionally the cell or plurality of cells, macrocellular structure or cardiocluster, or product of manufacture, are introduced into, onto or approximate to (close to) a mammalian heart, or are administered to or applied to an individual in need thereof.

The details of one or more embodiments as provided herein are set forth in the accompanying drawings and the description below. Other features, objects, and advantages of embodiments as provided herein will be apparent from the description and drawings, and from the claims.

All publications, patents, patent applications, GenBank sequences and ATCC deposits, cited herein are hereby expressly incorporated by reference for all purposes.

### BRIEF DESCRIPTION OF THE DRAWINGS

The patent or application file contains at least one drawing executed in color.

Copies of this patent or patent application publication with color drawing(s) will be provided by the Office upon request and payment of the necessary fee.

The drawings set forth herein are illustrative of embodiments as provided herein and are not meant to limit the scope of the invention as encompassed by the claims.

The figures are described herein:

FIG. 1A-D illustrate data showing that LIN28 marks a unique non-myocyte population in the heart:

FIG. 1A Graphically illustrates Flow cytometric analysis of LIN28 expression on mouse embryonic stem cells as positive control showing 92.9% positivity;

FIG. 1B Graphically illustrates Flow cytometric analysis of LIN28 expression on freshly isolated non-myocytes from dissociated murine heart samples showing 36.2% positivity;

FIG. 1C graphically illustrates Quantitation of flow cytometric assessment of Lin28 positivity in freshly isolated nonmyocyte population from the murine heart represented as fixed versus live cells preparations. The increase in positivity in the fixed sample is consistent with internal protein expression distinct from surface immunoreactivity;

FIG. 1D illustrates an image of a Confocal microscopy of an adult mouse heart section labeled for expression of LIN28 (red), myosin light chain 2 (teal), membranes (green, stained with wheat germ agglutinin (WGA)), and nuclei (blue, stained with DAPI). Note strong membrane immunoreactivity of interstitial cells in the section;

FIG. 1E illustrates an image showing that an adult FVB Mouse has distinct Lin28+ interstitial cell population. Three-month-old male FVB heart stained for Lin28 (green), ckit (red), cardiac troponin T (teal), WGA (grey) and nuclei (blue) demonstrate a distinct Lin28+ckit− cell population in situ;

FIG. 1F illustrates a human heart section (18 year old normal female) labeled for Lin28 (pink), myosin light chain (green), membrane (grey) and nuclei (blue).

FIG. 2A-C illustrate data showing that Lin28 expression increases in postnatal development: FIG. 2A illustrates an immunoblot analysis of heart lysates labeled for LIN28 showing two immunoreactive bands at 23.2 kDa and 21.0 kDa. Variation in protein loading corrected against either (3-actin or GAPDH; FIG. 2B illustrate two corresponding quantitations graphically illustrated in the two graphs demonstrate increasing protein expression over time after birth at multiple sampling points, corrections for each band (23.2 versus 21.0) relative to both loading controls (β-actin or GAPDH) are provided; FIG. 2C illustrates images of a confocal microscopy of an adult (8 week old) FVB mouse heart section labeled for expression of LIN28 (green), c-Kit (red), and membranes (white, stained with wheat germ agglutinin (WGA)).

FIG. 3 illustrates images of Lin28 expression at 6 weeks of age by confocal microscopy. Low (left panel) and high (right panel) magnification of murine heart section labeled for LIN28 (green), myosin light chain 2 (blue), membranes (red, stained with wheat germ agglutinin (WGA)), and nuclei (gray, stained with DAPI.

FIG. 4 illustrates images of Lin28 expression at embryonic day 13 by confocal microscopy. Murine heart section labeled for LIN28 (green), myosin light chain 2 (blue), membranes (red, stained with wheat germ agglutinin (WGA)), and nuclei (gray, stained with DAPI).

FIG. 5A-B illustrate images of LIN28 expression on c-kit+ expanded cardiac stem culture: FIG. 5A: Murine CPC (c-Kit+) line stained for LIN28 expression by flow cytometry and confocal microscopy, with the data graphically illustrated; FIG. 5B: Human CPC H16-110 line stained for LIN28 by flow cytometry and confocal microscopy, with the data graphically illustrated.

FIG. 6A-B illustrate images of LIN28+ stem cell culture testing with human and murine-derived cells: FIG. 6A (Upper left) Mouse LIN28+ isolated and cultured 3 days on gelatin in Elision media; FIG. 6A (Upper right) Mouse LIN28+ cultured line stained for LIN28 (red) and c-Kit (green); FIG. 6A (Lower left) Phase contrast microscopy for H16-114 cell line isolated using LIN28 at passage 8; FIG. 6A (Lower right): Immunolabeling for microscopy for H16-112 cell line isolated using LIN28 at passage 8; FIG. 6B graphically illustrates data showing Growth rate comparison between CPC versus LIN28 cultures for two different human isolates at passage 8; doubling time is approximately 50 to 60 hours.

FIG. 7 graphically illustrates a Flow cytometry assessment showing Surface marker characteristics for LIN28 and c-kit on freshly isolated nonmyocyte population; the Flow cytometry assessment shows overlap between double positive c-kit/LIN28 cells (6.16%), whereas the c-kit-/LIN28+ double positive cells account for 38.8% of the population.

FIG. 8A-B illustrates images showing: FIG. 8A illustrates an image showing the enlarged morphology of LIN28 cells co-cultured on neonatal rat cardiomyocytes 24 hours in to co-culture; LIN28 cells were tagged by overnight infection with GFP-expressing lentivirus and co-cultured with NRCM for 12 days; FIG. 8B illustrates an image showing that LIN28 cells show marked morphologic rearrangement and enlargement in the culture.

FIG. 9 illustrates images of unlabeled cryostat sections showing native fluorescence of adoptively transferred Lin28 cells: adoptively transferred LIN28 cells persist in the infarcted murine myocardium for at least 10 days following delivery; adoptively transferred LIN28 cells were detected by native fluorescence of GFP and mCherry fluorophores within the left ventricular wall of an infarcted murine myocardial section at 10 days post injection.

FIG. 10 illustrates images showing Lin28 and c-Kit cells localized in valve tissue of infarcted mouse heart. Paraffin sections of mouse hearts subjected to myocardial infarction for seven days were immunostained for c-Kit (red) and Lin28 (green). Inset shows nuclear (DAPI) and membrane (WGA) in white. White arrow indicates cell labeled for both c-Kit and Lin28.

FIG. 11 illustrates images showing Lin28 and c-Kit cells localized in infarcted mouse heart. Mouse heart sections were immunostained for c-Kit (red) and Lin28 (green). Inset shows nuclei (DAPI) and membrane (WGA) staining in white. White arrow indicates cell labeled for both Lin28 and c-Kit in infarcted region of myocardium.

FIG. 12 schematically illustrates an exemplary workflow for single cell RNA-sequencing. (A) Lin28+ cells and CPCs are cultured on gelatin-coated dish with Ellison's media and passaged up to approximately 5 to 7 passages. Single cells are captured by mouth-pipetting. (B) First-strand cDNA synthesis and cDNA amplification were done using Clontech SMART-Seq v4™ kit and cDNA library was tagmented using Illumina Nextera XT™ kit, and then subjected to be sequenced using Illumina NextSeq500™. (C) Sequencing data was mapped to mouse reference genome by STAR aligner™ and quantified using DESeq2™.

FIG. 13A-B illustrate a table of data, and graphically illustrates, a summary of the alignment of sequencing data; FIG. 13A: Average number of reads per cell is around 7 million and around 95% of the reads are aligned to mouse reference genome; FIG. 13B: Bar graph shows that sequence reads are aligned to different regions of the transcriptome and around 50% of reads are aligned to coding region. This summary data suggests that the single-cell RNA-sequencing was performed well for all of 11 Lin28+ cells and 5 CPCs.

FIG. 14: illustrates Table 1: showing Top Differentially Expressed Genes relatively abundant in Lin28+ cells or CPCs.

FIG. 15 illustrates data showing a Heatmap to represent differentially expressed genes between Lin28⁺ cells and c-Kit⁺ cells. 404 and 225 genes are enriched in c-Kit+ cells and Lin28+ cells, respectively.

FIG. 16 illustrates a table of data showing that Single Lin28+ cells are close to each other, and far from CPCs. Pearson's correlation coefficient between single cells were calculated based on the values for log 2(CPM+1). CPM: count per million reads.

FIG. 17 graphically illustrates data showing that Single Lin28+ cells are clustered close to each other and far from CPCs. To compare the transcriptomic similarity between Lin28+ cell and CPCs, principal component analysis was performed with 1938 genes, which are highly variable (coefficient of variation>1) and with high expression level (average FPKM>1). PCA plot shows a cluster of Lin28+ cells apart from CPCs regardless of its size.

FIG. 18 illustrates Table 2: showing Gene Set Enrichment Analysis (GSEA). To compare transcriptomic signature between Lin28+ cells and CPCs, GSEA was performed using Broad institute GSEA software. The table shows the representative gene sets enriched in CPCs or Lin28+ cells with high Normalized Enrichment Score (NES) and significant p-value/q-value. Representative gene sets are shown in FIG. 6 with GSEA plot and heatmap for leading edge genes.

FIG. 19A and FIG. 19B illustrates representative gene sets showing enrichment of differentially expressed genes either in Lin28+ cell (FIG. 19B) or CPCs (FIG. 19A).

FIG. 20A-D illustrates Canonical pathways showing different expression pattern between Lin28+ cells and CPCs. Each gene was color-coded based on gene expression data from RNA-seq using Ingenuity Pathway Analysis™. Several key components of JNK (FIG. 20A), ERK (FIG. 20B), mTOR (FIG. 20C), and Jak/STAT (FIG. 20D) signaling pathways are more abundant in CPCs comparing to Lin28+ cells, the CPC red to Lin28+ green imaging indication applies for FIG. 20A-D. [WE NEED CLEARER IMAGES, CANNOT READ THESE AS IS]

FIG. 21 illustrates a Compilation of essential RNA-Seq characteristics. (A) Heatmap of Lin28+ Interstitial Population cells (LIPs) versus cardiac progenitor cells (CPCs). (B) Venn diagram depiction that shows: 1) there are 10,431 distinct transcripts detected in LIPs, whereas there are 10,306 distinct transcripts detected in CPCs; 2) a total of 606 DEGs (differentially expressed genes) between the CPCs and LIPs representing approximately 6% of the transcriptome, 3) of the 606 DEGs there are 111 DEGs in LIPs that are not detected in CPCs (18.3% of DEGs), there are 28 DEGs in CPCs that are not expressed in LIPs (4.6% of DEGs), and there are 467 DEGs expressed in both CPCs and LIPs (77%) that show a difference of at least two fold change in expression that is statistically significant (p>0.05). (C) Volcano plot of DEGs represented between LIPs (red) versus CPCs (blue).

FIG. 22A-B illustrates LIP cell dynamics revealed by time-lapse video microscopy; two series of still frames of LIP cells with time stamp (upper left of each panel); the upper series of eight panels show a cell expressing FUCCI reporter entering mitosis as binucleated (green arrow, upper row third frame) indicated by green nuclear fluorescence reporter and after exit from mitosis showing red fluorescence giving rise to two mononucleated daughter cells; the lower series of eight panels shows a mononucleated cell (green arrow) giving rise to two mononucleated daughter cells (red arrows), as described in Example 1, below.

FIG. 23A-D illustrate that Lin28+ interstitial cells demonstrate upregulated RNA expression of secretome factors; FIG. 23A: qPCR array of adult mouse Lin28+ isolated cells passage 5 display higher RNA expression of secretome factors IGF and FGF2 compared to mouse CPCs passage 12; FIG. 23B-D: qPCR of HGF (FIG. 23B), IGF (FIG. 23C) and FGF2 (FIG. 23D) verify mLIN28 cells express higher secretome factors compared to mCPCs, bMSCs and CPCeKs.

FIG. 24A-B illustrate that Lin28 is expressed in cells from young and older human cardiac tissue and uniquely found in myocytes from older adults. Human heart tissue is stained for Lin28 (fushia), myosin light chain (green), WGA (grey) and nuclei (blue) in an 18 year old female (FIG. 24A) and 58 year old female (FIG. 24B), both without heart disease. The older adult unique demonstrates myocytes positive for Lin28.

FIG. 25 illustrates a schematic representation of an exemplary process used to isolate cardiac cells from LVAD tissue, as described in detail in Example 1, below.

FIG. 26A-B illustrates data showing that murine CPC aging correlates with diminished proliferation and lowered Lin28 expression: FIG. 26A graphically illustrates data showing the proliferation of CPCs isolated from young (3 month) versus aged (24 month) mouse hearts; FIG. 26B top panel illustrates an immunoblot of Lin28A and Lin28B, and FIG. 1B lower panel graphically illustrates a quantitation of the result of the immunoblot of FIG. 26B top panel, as described in detail in Example 1, below.

FIG. 27A-D illustrates LIP CD90 immunoselection yields distinct subpopulations. LIP subset properties revealed by confocal microscopy (FIG. 27A), CD90 expression (FIG. 27B), area (FIG. 27C) and average area for three human LIPs (FIG. 27D), as described in detail in Example 1, below.

FIG. 28A-D illustrates single cell RNA-Seq comparative analysis of LIP versus CPC: FIG. 28A illustrates a heatmap showing differentially expressed genes between LIP and CPC single cells; FIG. 28B illustrates a Venn diagram representation of differentially expressed (DE) genes; FIG. 28C illustrates a Volcano plot representing the top nine DE genes between CPC and LIP populations; FIG. 28D illustrates an tSNE population analysis demonstrating relationship between LIP (turquoise) versus CPC (salmon) populations, as described in detail in Example 1, below.

FIG. 29 illustrates an image of LIP expansion following infarction injury, as described in detail in Example 1, below.

FIG. 30 graphically illustrates data showing elevated stemness markers in LIPs, as described in detail in Example 1, below.

FIG. 31A-C graphically illustrate data showing that murine LIP co-culture improves proliferation and survival of murine CPCs: FIG. 31A graphically illustrate data showing the proliferation of LIP and mCPC in single culture and co-culture represented as a fold change relative to day of plating; FIG. 31B graphically illustrate data showing cell doubling time in hours; FIG. 31C graphically illustrate data of a cell death assay of mLIPS and mCPCs in single culture and co-culture after treatment with 80 μM H₂O₂, as described in detail in Example 1, below.

Like reference symbols in the various drawings indicate like elements.

Reference will now be made in detail to various exemplary embodiments of the invention, examples of which are illustrated in the accompanying drawings. The following detailed description is provided to give the reader a better understanding of certain details of aspects and embodiments as provided herein, and should not be interpreted as a limitation on the scope of the invention.

### DETAILED DESCRIPTION

In alternative embodiments, provided are isolated Lin28+ cardiac stem cells, including Lin28+ adult cardiac stem cells and Lin28+ cardiac progenitor cells, and Lin28+ cells as isolated using methods as provided herein, and methods for making (for isolating them) and using them. Also provided are methods for inducing or accelerating cardiogenesis in the mammalian (e.g., a human) heart, or for treating a heart genetic defect, injury or dysfunction, or an injury or dysfunction subsequent to (or following, or optionally from 1 minute to 12 hours after, or immediately after) an ischemic injury or a heart failure, or an injury or dysfunction resulting from a myocardial infarction (MI) (in an individual in need thereof), comprising administering to an individual one or more of the Lin28+ cardiac stem cells or cardiac progenitor cells as provided herein, including Lin28+ cardiac stem cells or cardiac progenitor cells isolated by a method as provided herein, or a Lin28+ interstitial population (LIP) provided herein, or administrating to a patient a product of manufacture provided herein.

Screening of embryonic stem cell antigen markers identified a Lin28+ interstitial population (LIP) that possesses characteristics distinct from classically studied cardiac stem cell archetypes. Lin28 is an embryonic stem cell antigen associated with cellular pluripotency, and heretofore (before this invention) was unrecognized as a stem cell marker in the adult myocardium.

In alternative embodiments, provided are methods for using a Lin28+ interstitial population (LIP), or Lin28+ cardiac stem cell population, both as provided herein, both populations having an inherently enhanced potential for mitigating myocardial damage for mitigating myocardial damage, and for treating or ameliorating a heart injury, an injury subsequent to (or following, or optionally from 1 minute to 12 hours after, or immediately after) a myocardial infarction (MI), a congenital or genetic heart defect, or a heart dysfunction. Results using both human and murine samples confirm unique phenotypic properties of exemplary LIPs as provided herein relative to established cardiac stem cell categories (e.g. cardiac progenitor cells (CPC), mesenchymal stem cells (MSC), and endothelial progenitor cells (EPC)). Moreover, exemplary LIPs as provided herein exhibit remarkable persistence and engraftment following adoptive transfer into infarcted myocardium, thus demonstrating their use to blunt myocardial injury and mediate myocardial repair. In alternative embodiments, provided are methods comprising an adoptive transfer of culture-expanded LIPs to a patient in need thereof to treat or ameliorate a heart injury, an injury subsequent to (or following, or optionally from 1 minute to 12 hours after, or immediately after) a myocardial infarction (MI), a congenital or genetic heart defect, or a heart dysfunction. In one embodiment, exemplary LIPs, or Lin28+ cells, as provided herein are injected into a border zone of the ventricular wall of hearts, e.g., cells are injected into infarcted myocardium.

Described herein for the first time is the identification and isolation, with novel uses, of these LIP novel cell populations that has a regenerative potential relative to alternative cardiac stem cell populations, including CPC, EPC, and MSC, and provides methods for using a Lin28+ interstitial population (LIP), or Lin28+ cardiac stem cell population, in cardiac regenerative medicine for e.g., myocardial healing.

In alternative embodiments, provided are macrocellular structures or an artificially configured plurality of cells, or so-called “cardioclusters”, e.g., as described in US 2016-0166617 A1, comprising Lin28+ cardiac stem cells, including Lin28+ cardiac stem cells as provided herein, including Lin28+ cells as isolated using methods as provided herein.

In alternative embodiments, the macrocellular structures or artificially configured plurality of cells, the so-called “cardioclusters” as provided herein (comprising Lin28+ cardiac stem cells, including Lin28+ cells as isolated using methods as provided herein), can simulate the “natural” stem cell microenvironments in which communities of cells of different types exist in an organized relationship.

In alternative embodiments, exemplary Lin28+ cardiac stem cells as provided herein, including Lin28+ cardiac stem cells, including Lin28+ cardiac stem cells as provided herein, including Lin28+ cells as isolated using methods as provided herein, or the Lin28+ macrocellular structures or artificially configured plurality of cells, the so-called “cardioclusters” as provided herein, provide a milieu for stem cell self-renewal and differentiation, which can be tightly controlled in defined locations of all regenerative tissues, including the heart. In alternative embodiments, functions of microenvironments re-created by exemplary Lin28+ cardiac stem cells, including exemplary Lin28+ cardiac stem cells as provided herein, including exemplary Lin28+ cells as isolated using methods as provided herein, or by macrocellular structures as provided herein, include the maintenance, or stimulation, of a quiescent stem cell population that are hypersensitive to stimuli such as molecular signaling and extracellular matrix (ECM) remodeling. In alternative embodiments, exemplary Lin28+ cardiac stem cells, including exemplary Lin28+ cardiac stem cells as provided herein, including exemplary Lin28+ cells as isolated using methods as provided herein, or the macrocellular structures as provided herein, can create cardiac niches that can regulate symmetric or asymmetric stem cell division, as asymmetric division of CPCs creates new cardiogenic daughter cells with properties required to repopulate the damaged myocardium. In alternative embodiments, exemplary Lin28+ cardiac stem cells, including exemplary Lin28+ cardiac stem cells as provided herein, including exemplary Lin28+ cells as isolated using methods as provided herein, or macrocellular structures as provided herein, can recapitulate, or recreate, cardiac microenvironments in vivo or ex vivo, and create an enhanced cellular communication, e.g., by expression of the gap junction protein connexin 43, improving cell propagation and differentiation in vitro.

In alternative embodiments, exemplary Lin28+ cardiac stem cells, including exemplary Lin28+ cardiac stem cells as provided herein, including exemplary Lin28+ cells as isolated using methods as provided herein, or the macrocellular structures as provided herein, can generate paracrine effects, for example, they can restore vasculature via endothelial precursor cells. In alternative embodiments, exemplary Lin28+ cell macrocellular structures as provided herein are rationally designed to promote efficient cardio-myogenesis.

In alternative embodiments, exemplary Lin28+ cell macrocellular structures as provided herein are formed by directed, or random, aggregation, e.g., on a matrix, e.g., an extracellular matrix (ECM), leading to variable sphere size. In alternative embodiments, exemplary Lin28+ cell macrocellular structures as provided herein can be designed to have consistent cell characterization markers, thus making the administration and treatment protocols and effects reproducible for everyday clinical practices.

In alternative embodiments, exemplary Lin28+ cardiac stem cells, including exemplary Lin28+ cardiac stem cells as provided herein, including exemplary Lin28+ cells as isolated using methods as provided herein, or the macrocellular structures as provided herein, are used in cellular therapy, and for the administration or application of stem cell populations to treat cardiovascular diseases, and to promote (cause to happen) efficient cardiac regeneration, or to stimulate or initiate cardiac regeneration. In alternative embodiments, instead of introducing just a single cell type in an unnatural context, exemplary Lin28+ macrocellular structures as provided herein can restore multiple cell types simultaneously, where the cells are already organized in a manner that mimics their natural environment, and exemplary Lin28+ macrocellular structures as provided herein can initiate or facilitate (e.g., accelerate) regeneration of heart tissue that has been damaged as a consequence of cardiac disease or injury.

In alternative embodiments, exemplary Lin28+ cardiac stem cells, including exemplary Lin28+ cardiac stem cells as provided herein, including exemplary Lin28+ cells as isolated using methods as provided herein, or the exemplary Lin28+ macrocellular structures as provided herein, are used to replace (and can replace) damaged tissue, necrotic tissue, or scar tissue in the mammalian heart (e.g., a human heart) with functional cardiac cell populations normally found within the myocardium.

In alternative embodiments, exemplary Lin28+ cardiac stem cells and Lin28+ macrocellular structures as provided herein are used to deliver beneficial stem cells or precursor cells, such as MSCs having the ability to secrete a diverse assortment of paracrine factors, to a tissue in need thereof, e.g., to a heart. In alternative embodiments, exemplary Lin28+ macrocellular structures as provided herein deliver EPCs to form microvessels, and thus are also used to support, accelerate or initiate blood vessel maturity. EPCs and MSCs delivered (e.g., by implantation into or near a tissue, e.g., a heart) by exemplary Lin28+ macrocellular structures as provided herein can have diverse properties and regeneration potential, and achieve long-lasting myocardial benefits that require the interaction of multiple cell types. In alternative embodiments, exemplary Lin28+ macrocellular structures can provide multiple stem cells types, in some embodiments, cells explanted from the human heart. In alternative embodiments, exemplary Lin28+ macrocellular structures as provided herein can provide CPCs that are pre-committed to the cardiovascular lineage and thus can produce new cardiogenic cells without inducing arrythmyogenesis, a distinct advantage over other cell types for cardiac cell therapy.

In alternative embodiments, exemplary Lin28+ cardiac stem cells, including exemplary Lin28+ cardiac stem cells as provided herein, including exemplary Lin28+ cells as isolated using methods as provided herein, or exemplary Lin28+ macrocellular structures as provided herein, provide the regenerative potential of stem cells in a clinical setting, e.g., providing stem cells capable of performing a variety of mechanisms for myocardial repair. In alternative embodiments, different exemplary Lin28+ cardiac stem cells and macrocellular structures as provided herein are rationally designed and based on known characteristics and functions of CPCs, MSCs and EPCs. In alternative embodiments, exemplary Lin28+ cardiac stem cells, including Lin28+ cardiac stem cells as provided herein, including exemplary Lin28+ cells as isolated using methods as provided herein, or exemplary Lin28+ macrocellular structures as provided herein, can deliver to a patient (e.g., an individual in need thereof) cardiogenic stem cells for ameliorating, accelerating the healing of, or treating a diseased heart, or slowing the progress of heart disease. In alternative embodiments, exemplary Lin28+ cardiac stem cells and exemplary Lin28+ macrocellular structures as provided herein can recapitulate the complex network found within stem cell microenvironments.

### Methods for Administering Lin28+ Cells and Lin28+ CardioClusters

In alternative embodiments, exemplary Lin28+ cardiac stem cells as provided herein, including exemplary Lin28+ cells as isolated using methods as provided herein, or the exemplary Lin28+ macrocellular structures as provided herein (“Lin28+ cardioclusters”), are administered (e.g., to an individual in need thereof) to induce or accelerate cardiogenesis in a mammalian (e.g., a human or a non-human, e.g., animal) heart, or for treating or ameliorating a heart injury, a congenital or genetic heart defect, or a heart dysfunction. In alternative embodiments, exemplary Lin28+ cardiac stem cells as provided herein, including exemplary Lin28+ cells as isolated using methods as provided herein, or the exemplary Lin28+ macrocellular structures as provided herein (“Lin28+ cardioclusters”), can be administered by any means known in the art, for example, by local injection (including e.g., intracoronary, intramyocardial and endocardial routes), infusion, use of implants, stents or catheters, or equivalent techniques. In alternative embodiments, so-called “cardioclusters” as provided herein are administered, or delivered in vivo, through coronary arteries, coronary veins, or peripheral veins; or, alternatively, via direct intramyocardial injection using a surgical, transendocardial, or transvenous approach; see e.g., Rosen et al., J Am Coll Cardiol. 2014; 64(9):922-937; Perin et al. Nat Clin Pract Cardiovasc Med. 2006 March; 3 Suppl 1:S110-3. In one embodiment, catheters are used to administer, or deliver, exemplary Lin28+ cardiac stem cells as provided herein, including exemplary Lin28+ cells as isolated using methods as provided herein, or exemplary Lin28+ macrocellular structures as provided herein (“Lin28+ cardioclusters”), e.g., ND INFUSION CATHETER™ Translational Research Institute, aka TRI Medical (Frankfurt, Germany).

Techniques for delivering nucleic acids (e.g., gene therapy) to the heart can also be adapted for administration of exemplary Lin28+ cardiac stem cells as provided herein, including exemplary Lin28+ cells as isolated using methods as provided herein, or exemplary Lin28+ macrocellular structures as provided herein (“Lin28+ cardioclusters”). Examples and descriptions of such protocols and techniques that can be used, and adapted, to practice compositions and methods as provided herein in vivo are e.g., Rasmussen (2011) Circulation, vol 123, pgs 1771-1779; Bridges et al., Annals of Thoracic Surgery, 73: 1939-1946 (2002); Wang, et al., Catheter, Circulation, 2009, pp. S238-S246, vol. 120, suppl. 1. Vulliet, et al., Lancet, Mar. 6, 2004; WO 2005/030292 (Apr. 7, 2005); WO 2005/027995; U.S. Patent application publication 20060258980; U.S. Pat. Nos. (USPNs) 7,722,596; 8,158,119; 8,846,099.

Additionally, materials or delivery adjuvants can be used to enhance cell retention and their longevity once delivered to a heart, e.g., by administration with or formulated with (e.g., mixed with) a gel or a hydrogel, such as a chitosan-based hydrogel, e.g., as described in Kurdi et al. Congest Heart Fail. 2010 May-June; 16(3):132-5, or any biocompatible scaffold, e.g., as described in U.S. Pat. Nos. 8,871,237; 8,753,391; 8,802,081; 8,691,543, or Pagliari et al. Curr Med Chem. 2013; 20(28):3429-47, or biomimetic support, e.g., as described in Karam et al. Biomaterials. 2012 August; 33(23):5683-95.

### 3-D Printing and Three-Dimensional Living Biological Tissue

Provided are three-dimensional synthetic, semi-synthetic, or living biological tissues made using (or comprising) exemplary Lin28+ cardiac stem cells as provided herein, including exemplary Lin28+ cells as isolated using methods as provided herein, or exemplary Lin28+ macrocellular structures as provided herein (“Lin28+ cardioclusters”). In one embodiment, exemplary Lin28+ cardiac stem cells or Lin28+ cardioclusters are used in a “bio-printing” process to generate a spatially-controlled cell pattern using a 3D printing technology. Any bio-printing or bio-fabricating process known in the art can be used, e.g., as described in U.S. Pat. App. Pub. Nos. 20140099709, 20140093932, 20140274802, 20140012407, 20130345794, 20130190210 and 20130164339; and U.S. Pat. No. 8,691,974.

For example, in one embodiment, a printer cartridge is filled with a suspension of exemplary Lin28+ cardiac stem cells or “Lin28+ cardioclusters” as provided herein and a “smart gel”; the, alternating patterns of the smart gel and cells or cardioclusters are printed using a standard print nozzle. In alternative embodiment, a NovoGen (San Diego, Calif.) MMX™, or Organovo Holdings, Inc., bioprinters are used for 3D bioprinting. This and equivalent “bio-printers” can be optimized to “print”, or fabricate, skin tissue, heart tissue, and blood vessels, and other basic tissues, all of which are suitable for surgical therapy and transplantation.

### Kits

Provided are kits comprising compositions as provided herein and methods as provided herein, including exemplary Lin28+ cardiac stem cells as provided herein, or exemplary Lin28+ cells as isolated using methods as provided herein, or exemplary Lin28+ macrocellular structures as provided herein (“Lin28+ cardioclusters”). As such, kits, cells, instructions and the like are provided herein.

The invention will be further described with reference to the following examples; however, it is to be understood that the invention is not limited to such examples.

### Example 1: Isolating Lin28+ Cardiac Stem Cells

The following example describes an exemplary method for isolating Lin28+ cardiac stem cells, as provided herein:

### Human LIN28⁺ Interstitial Population

Cardiac biopsies were obtained from patients undergoing LVAD implantation. NIH guidelines for human subject research are consistent with Institutional Review Board (IRB) exemption based upon the use of tissues that are waste discards from normal and routine clinical procedures of LVAD surgery (45 CFR 46.101). After excision, cardiac tissue remained on ice in cardioplegic solution until processed. Fatty tissue was excised and remaining cardiac tissue was suspended in Basic Buffer (15 ml) and minced into 1 mm³ pieces. After mincing, tissue and Basic Buffer were collected in 50 ml Falcon tube. Digestive solution containing collagenase, type II 225 U/mg dry weight (Worthington, catalog #LS004174, Bio Corp, Lakewood, N.J.) was dissolved in Basic Buffer (2-2.5 mg/ml) and incubated with tissue pieces for 1.5-2 hours at 37° C. with continuous shaking. Digestion solution was refreshed at the one-hour time point and resulting suspensions were centrifuged at 350 g and resuspend in cardiac stem cell media (see Table 1). Final suspension was filtered through a 100-μm filter (Corning, Inc., catalog #352360) followed by a 40-μm filter (Corning, Inc., catalog #352340) and centrifuged at 150 g for 2 minutes to collect CMs. The supernatant was collected and centrifuged at 350 g and resuspend in incubation buffer.

To isolate LIN28⁺ cells, suspension was incubated with LIN28⁺ polyclonal rabbit antibody (ThermoFischer, PA1-096) at a 1/10 dilution per 1×10⁶ cells for 30 minutes at 4° C. rocking.

After incubation, 5 mL of wash buffer was added and suspension was centrifuged at 350 g for 5 minutes to remove any unbound primary antibody. Remaining cell pellet was resuspend in incubation buffer with a 1/50 dilution of biotin donkey anti-rabbit (Jackson ImmunoRes, catalog #711-065-152) secondary antibody for 15 minutes rocking at 4° C. Once again 5 mL of wash buffer and centrifugation was used to remove any unbound secondary. Cells were then re-suspended in incubation buffer and Streptavidin MicroBeads for a dilution of 1/50 for 15 minutes rocking at 4° C. a final wash step was performed to remove any unbound beads prior to column sorting. Using the QuadroMACS system, the non-CM cell suspension plus antibody-bio/strep-bead tagging was run through LS columns. Columns were washed with at least 8 mL of wash buffer prior to collecting positive fraction. LIN28⁺ cells were centrifuged at 350 g for 5 minutes prior to plating on 6 mm cell culture dish in cardiac stem cell medium.

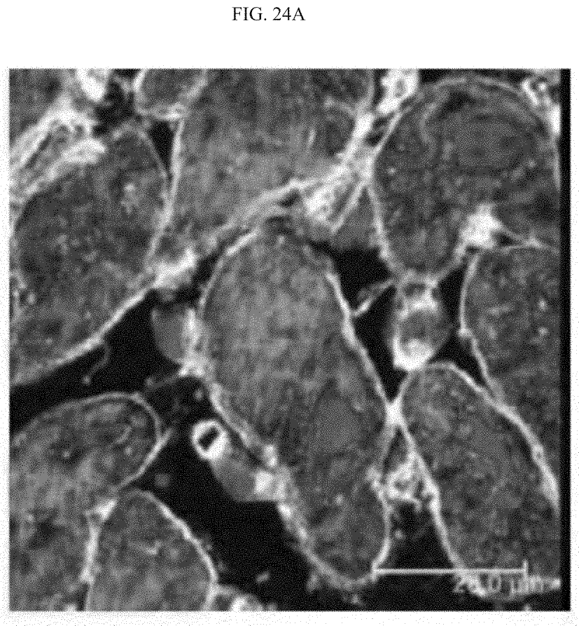

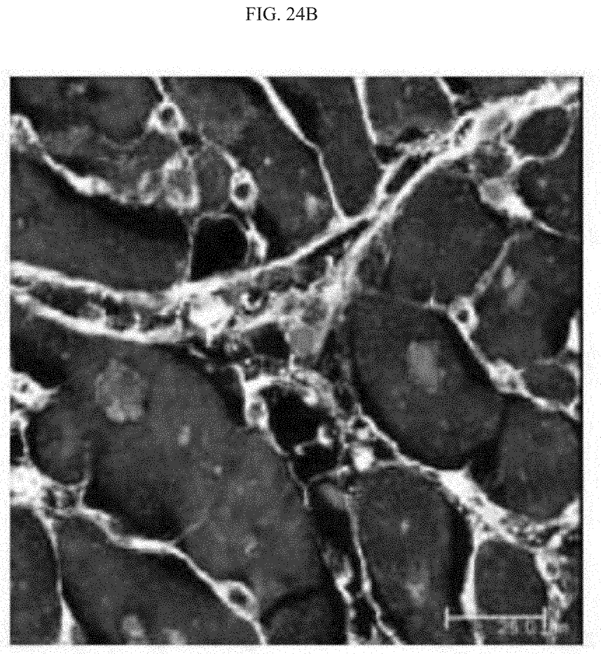

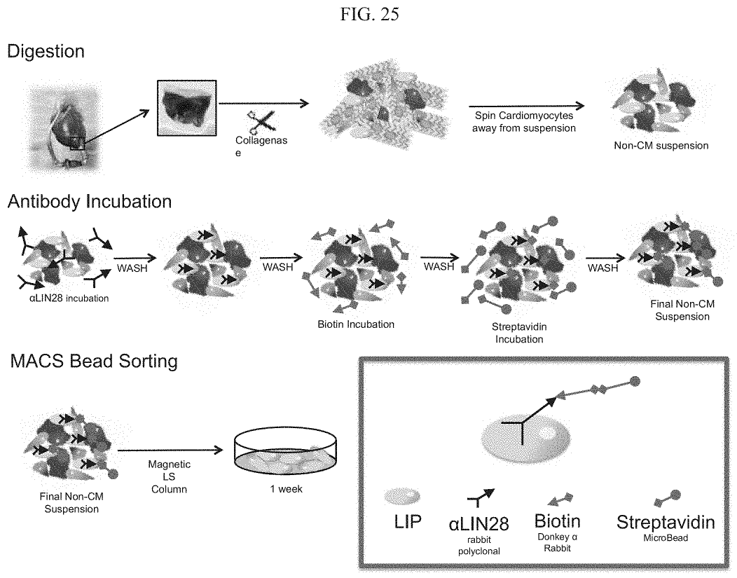

### FIG. 25. Lin28⁺ Interstitial Progenitor Isolation Protocol

FIG. 25 illustrates a schematic representation of an exemplary process used to isolate cardiac cells from LVAD tissue. Following open-heart surgery, the tissue plug is digested to the single cell level and, following centrifugation to remove the CMs, plated overnight at 37° C. in CO₂ incubator. The single cell suspension is incubated with primary LIN28 antibody followed by incubation with Biotin and finally a Streptavidin MicroBead (inserted box). Magnetic bead isolation through LS columns purify the population resulting in LIPs. Yellow cells indicate LIPs; Square pink stripped cells represent cardiomyocytes; Red, Blue and Green cells represent the heterogeneity of the interstitial population, non-CM.

### Materials

### Reagents:

Phosphate-Buffered Saline (PBS 1×; Corning™, cat. no. 21-040-CV)
J-MEME (Joklik Modification, with L-glutamine, without calcium chloride and sodium bicarbonate, suitable for cell culture; Sigma™, cat. no. MO518)
HyPure™ Cell Culture Grade Water Endotoxin Free (Fischer™ cat. no. SH30529.03)
HEPES (Sigma, cat. no. H3375)
PSG (Penicillin-Streptomycin-Glutamine (100×); GIBCO™ cat. no. 10378016)
Taurine (Sigma cat. no. T8691)
Insulin from bovine pancreas (solvate in 3% acetic acid/PBS; Sigma™, cat. no. I-5500)
Amphotericin B (GIBCO, cat. no. 15290018)
Gentamicin Solution (Sigma cat. no. G1397)
Collagenase, Type 2 (approximately 225-320 u/mg; Worthington™ cat. no. CLS-2)
Bovine Serum Albumin (fatty acid free, low endotoxin, lyophilized powder, BioReagent™, suitable for cell culture, ≥96%; Sigma cat. no. A8806)
HyClone Ham's Nutrient Mixture F10-F12 media (Fischer™ cat. no. SH30026.01)
Embryonic Stem Cell Fetal Bovine Serum (ES FBS; GIBCO™ cat. no. 10439)
L-Glutathione reduced (Sigma™, cat. no. G6013)
Erythropoietin Human, Recombinant, CHO Cells (hEPO; EMD Millipore™ cat. no. 329817)
Recombinant Human FGF-basic (bFGF; Peprotech™ cat. no. 100-18B)
Cellstripper™ (Corning, catalog #25-056-CI)
TrypLE Express™ (1×) (Thermo Fisher Scientific™, catalog #12604-013)
Streptavidin MicroBeads (Miltenyi™, cat. no. 130-048-101
FcR Blocking Reagent, human (Miltenyi™, cat. no. 130-059-901)
Biotin-SP AffiniPure™ Donkey Anti-Rabbit IgG (Jackson ImmunoRes™ cat no. 711-065-152)
LIN28A Polyclonal Antibody (ThermoFischer™ cat. no. PA1-096)

### Equipment

Delicate scissors (Fine Science Tools™)
Forceps (Fine Science Tools™)
15-ml centrifuge tubes (Corning, cat. no. 430791)
50-ml centrifuge tubes (Corning™, cat. no. 430829)
Cell culture dish, 150 mm (Olympus™, cat. no. 715001)
Cell culture dish, 21.2 cm³ (Olympus™, cat. no. 25-260)
Sterile 50 mL Disposable Vacuum 0.22 μm filter (Millipore™, cat. no. SCGP00525)
Millipore Express PLUS™ Funnel 500 mL 0.22 μm filter (Millipore™, SCGPU05RE)
5-ml pipette (Corning, cat. no. CLS4050)
Rotating incubation chamber (Fischer Scientific™, Isotemp™)
Falcon Cellstrainer™ 40 um (Corning™, cat. no. 352340)
Falcon Cellstrainer™ 100 um (Corning™, cat. no. 352360)
5-ml syringe
Parafilm
Centrifuge, type 5810R (Eppendorf)
1.5-ml microcentrifuge tubes (Eppendorf)
QuadroMACS Starting Kit™ (LS) (Miltenyi cat. no. 130-091-051)
   QuadroMACS™ Separator (130-090-976)
   MACS MultiStand™ (130-042-303)
LS columns (Miltenyi™ cat. no. 130-042-401)

### Reagent Set-Ups

Basic Buffer:

Dissolve 11.2 g of J-MEM, 1.26 g of Taurine, and 0.7 g of HEPES in approximately 800 mL of cell culture grade water. Adjust final pH to 7.2-7.3 using NaOH. Move solution to sterile filter hood and add remaining reagents: 20 U Insulin, 10 mL PSG, 10 mL Amphotericin B and 50 uL Gentamicin. Adjust solution to 1 L using cell culture grade water and sterile filter in 0.2 um filter.

Collagenase Solution:

Dissolve 100 mg of Collagenase Type 2 to a final concentration range of 10-12 U/mL in a 50 ml conical tube. Sterile filter using 50 mL SteriFlip™.

Wash Buffer:

Dissolve 0.25 g of BSA into 50 mL of PBS for a final concentration of 0.5% BSA/PBS wash buffer.

Cardiac Progenitor Cell Culture Medium (CPC Medium):

Ham's F12 base medium containing 10% ES FBS, 0.2 mM L-Glutathione, 5 mU/mL human Erythropoietin, 10 ng/mL bFGF and 1% PSG. Medium can be stored at 4° C. for up to one month. Incubation buffer for cell isolation: 2 mL 0.5% BSA/PBS wash buffer with 200 uL of human FcR block (final dilution 1/100).

### Methods

### Human LIN28⁺ Interstitial Population

### Creating a Single-Cell Suspension (Approximate Timing 3.5-4 Hours)

1. Obtain cardiac biopsy from patient undergoing LVAD implantation. Biopsy is stored in cardioplegic solution and on ice until processed.

=>All procedures should be performed in sterile conditions from this point moving forward.

2. Place tissue in 150 mm cell culture dish with at least 5 mL of warmed (37 C) Basic Buffer. Carefully remove any large areas of fatty tissue form the sample and discard.

3. Mince tissue into small pieces ˜1 mm³ in size using forceps and scissor and then transfer minced tissue and solution using a wide bore 10 mL pipette into a 50 mL conical tube.

4. Add 15 mL of 2× Digestive Solution to the minced tissue and Basic Buffer. Total volume of tube is 30 mL.

=>secure tube with Parafilm if incubator is not sterile.

4. Place tube in rotating incubator set at 37° C. about 5 rotations/minute for 1.5-2 hours.

5. Remove tube and centrifuge at 350 g to remove collagenase solution.

6. Resuspend whole tissue pellet in 30 mL of CPC media. Gently pipette up and down to continue breaking apart any undigested pieces.

=>note that not all tissue may be digested and some small chunks may remain.

7. Filter solution through 100 um filter. Rinse filter with wash buffer (1-2 mL).

=>use syringe plunger to mash remaining chunks on filter. Some tissue will be left behind and multiple filters can be used if clogging occurs.

8. Filter solution through 40 um filter. Rinse filter with wash buffer (1-2 mL).

9. Centrifuge cell suspension at 150 g for 2 minutes to collect CMs.

10. Remove and KEEP supernatant in separate tube. Store CM pellet for RNA or protein collection. Pellet can be split to obtain both RNA and protein from CMs.

11. Centrifuge supernatant (non-CM cell population) at 350 g for 5 minutes. Gently aspirate supernatant from cell pellet.

=>The whole non-CM cell suspension can be plated on a 150 mm dish in cardiac stem cell medium over night at 37° C. in cell culture CO₂ incubator. OR proceed directly into antibody incubation

### Isolation of LIPs (Approximate Timing 1.5-2 Hours)

If non-CM cell suspension was plated overnight proceed with removing cells from the plate. If not proceed to step 12. Removing cells from plate: COLLECT medium in 50 mL conical tube. Wash plate with PBS and strip using disassociation buffer for 5 minutes in cell culture CO₂ incubator. Collect the cell suspension and add to collected medium. Wash dish with 3 mL of CPC medium and collect. Centrifuge cell suspension to remove CPC medium and disassociation buffer. Centrifuge cell suspension at 350 g for 5 minutes to obtain non-CM pellet.

12. To isolate LIN28⁺ cells, resuspend non-CM pellet in 15 mL conical tube with 300 uL of incubation buffer and add 30 ul of LIN28 polyclonal antibody (per approximately 1×10⁶ cells).

13. Secure conical tube with Parafilm and place tube on rocker at 4° C. for 30 minutes.

14. Add 5 mL of wash buffer to cell and antibody suspension and centrifuge at 350 g for 5 minutes.

15. Resuspend non-CM pellet in 300 uL of incubation buffer and add 15 uL of Biotin Donkey anti-Rabbit secondary antibody.

16. Secure conical tube with Parafilm and place tube on rocker at 4° C. for 15 minutes.

17. Add 5 mL of wash buffer to cell and antibody suspension and centrifuge at 350 g for 5 minutes.

18. Resuspend non-CM pellet in 300 uL of incubation buffer and add 15 uL of Streptavidin MicroBeads.

19. Secure conical tube with Parafilm and place tube on rocker at 4° C. for 15 minutes.

20. Add 5 mL of wash buffer to cell and antibody suspension and centrifuge at 350 g for 5 minutes.

21. Resuspend pellet in 500 uL-1 mL of wash buffer.

22. Set up MACS magnetic collection with QuadroMAC™ magnet and LS columns. Rinse columns in 5 mL of wash buffer prior to adding sample. (Can collect negative non-CM suspension for further isolations if desired).

23. Add sample to column and run through completely.

24. Add 8 mL of wash buffer to column to wash any negative cells out of column.

25. Remove LS column from magnet and place over new 15 mL conical tube. Add 5 mL of wash buffer to column and plunge cells from column. This is the LIN28⁺ cell suspension.

26. Centrifuge LIN28⁺ cell suspension at 350 g for 5 minutes.

27. Plate cells on 6 mm dish in CPC medium and place at 37° C. in cell culture CO₂ incubator.

28. Let cells grow to approximate 60-70% confluence before disassociation and re-plating.

TABLE 1
List of Media

| | Component | Catalog Number |
|---|---|---|
| Cardiac | F12 HAM's (1x) | SH30026.01, HyClone ™ |
| Stem Cell | 10% ES FBS | 16141079, Gibco ™ |
| Medium | 1% Penicillin- | 10378016, Gibco ™ |
| | Streptomycin- | |
| | Glutamine (100X) | |
| | 5 mU/mL human | E5627, Sigma Aldrich ™ |
| | erythropoietin | |
| | 10 ng/mL human | HRP-0011, Biopioneer ™ |
| | recombinant basic | |
| | FGF | |
| | 0.2 mM L-Glutathione | 66013-256, Sigma Aldrich ™ |
| Basic | 11 g/L Minimum | M0518, Sigma Aldrich ™ |
| Buffer | Essential Medium | |
| | Eagle, Joklik | |
| | Modification | |
| | 3 mM HEPES | H3375, Sigma Aldrich ™ |
| | 1% Penicillin- | 10378-016, Gibco ™ |
| | Streptomycin- | |
| | Glutamine (100X) | |
| | 10 mM Taurine | T0625, Sigma Aldrich ™ |
| | Insulin, solvate in | I-5500, Sigma Aldrich ™ |
| | 3% Acetic Acid/PBS | |
| | 1% Amphotericin B | 15290-018, Invitrogen ™ |
| | 50 mg Gentamicin | G1397, Sigma Aldrich ™ |
| | Cell Culture Grade Water | SH30529.03, Fischer ™ |
| Collagenase | Basic Buffer | Made above |
| Solution | Collagenase Type A | CLS-2, Worthington ™ |
| Wash | Bovine Serum | A8806, Sigma ™ |
| | Albumin | |
| Buffer | PBS | 21-040-CV, Corning ™ |
| Incubation | Wash Buffer | Made Above |
| Buffer | FcR Blocking Reagent, | 130-059-901, Miltenyi ™ |
| | human | |

TABLE 2
List of antibodies

| | | Catalog | Dilution |
|---|---|---|---|
| Antibody | Vendor | Number | Isolation |
| LIN28 A | Thermo Fischer ™ | PA1-096 | 1:10 |
| Anti-Biotin | Jackson | 711-065-152 | 1:50 |
| | ImmunoResearch ™ | | |
| Streptavidin | Miltenyi ™ | 130-048-101 | 1:50 |
| Mircobead | | | |

LIP Exists in Proximity to, but Distinct from, Traditional c-Kit+ CPCs

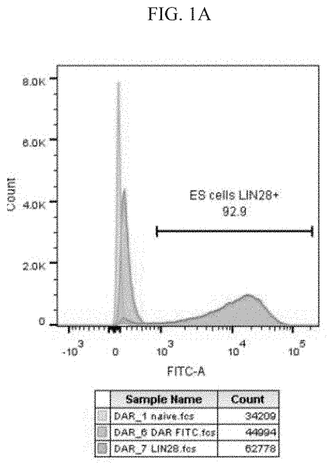

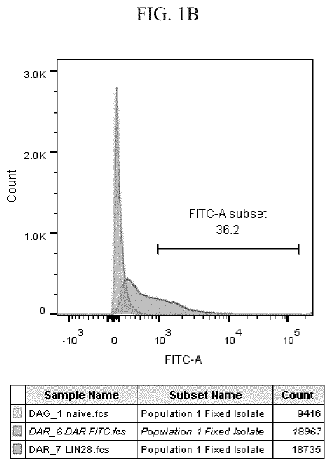

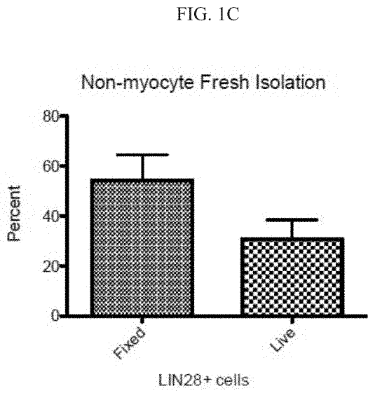

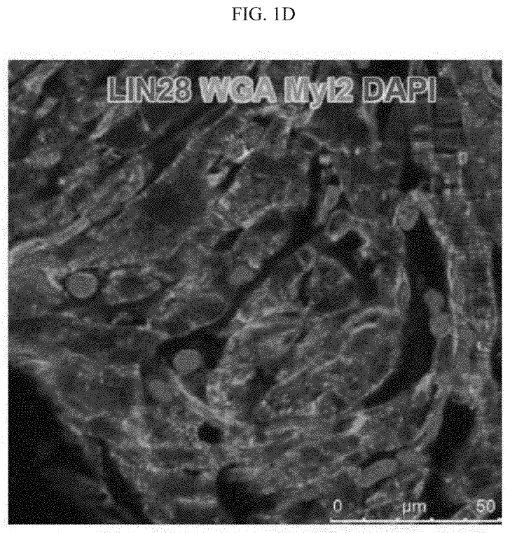

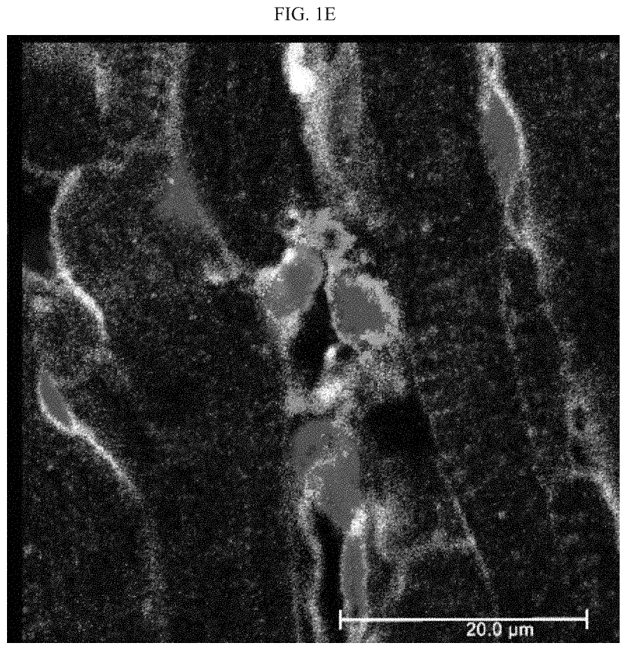

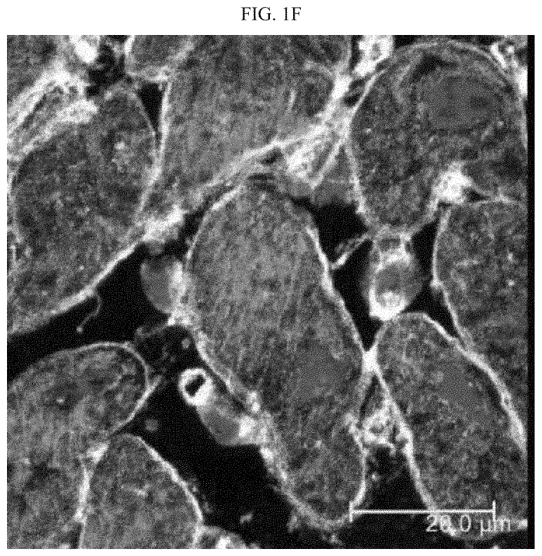

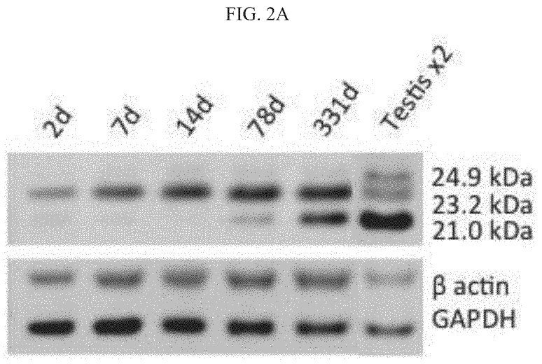

Initial efforts were focused upon identification and localization of LIP in myocardial tissue sections of human and murine origin to assess their relationship to c-kit+ CPCs that have been the topic of substantial debate.⁴⁶⁻⁴⁸ Reassuringly, immunofluorescent visualization of LIP follows standard protocols without need for signal amplification or enhanced detection procedures often required for visualization of endogenous c-kit expression.⁴⁹⁻⁵¹ As expected, in the sections from murine heart the LIP presented as small (less than (<) 20 μM diameter) interstitial cells with a large nucleus and minimal cytoplasm typical of cardiac stem cells (FIG. 1D). Furthermore, LIP localization was often in proximity to but distinct from c-kit+ immunolabeling indicating that LIP was distinct from CPCs (FIG. 1E) and c-kit+/Lin28+ cells were rarely observed. Presence of LIP in human myocardial samples recapitulating findings from the mouse results was confirmed using control non-pathological cardiac tissue sections showing comparable size and location (FIG. 1F). These results prompted further characterization of LIP to define basic biological parameters.

FIG. 1 illustrates LIP confocal microscopy localization with interstitial cell labeling: FIG. 1D illustrates an adult FVB murine heart section labeled for Lin28 (red, or darker spots), myosin light chain (teal), membrane (green), and nuclei (blue); FIG. 1E illustrates an adult FVB murine heart labeled for Lin28 (green), c-kit (red), cardiac troponin T (teal), membrane (white), and nuclei (blue); FIG. 1F illustrates a human heart section (18 year old normal female) labeled for Lin28 (pink), myosin light chain (green), membrane (grey) and nuclei (blue). Scale bar as indicated.

### LIP Comprise a Substantial Percentage of the Non-Myocyte Cells.

Freshly isolated cell suspensions obtained from human heart tissue were processed to enrich for the nonmyocyte fraction and remove cardiomyocytes as well as cell debris prior to immunolabeling with Lin28 antibody with subsequent (or following, or optionally from 1 minute to 12 hours after, or immediately after) flow cytometric analysis. With stringent gating, LIP was readily detected within the nonmyocyte population as a substantial fraction of the cells (FIG. 1B). Since Lin28 function as a cytoplasmic protein, samples were compared between live versus fixed cell conditions for LIP fraction. Consistent with prior studies showing differential c-kit positivity based upon fixation conditions⁵², the fraction of Lin28+ cells increased from 32% for live cells to 60% for fixed cells (FIG. 1C).

FIG. 1B-C: LIP frequency in freshly isolated CIC by flow cytometry: FIG. 1B illustrates a plot showing unlabeled cells (brown), FITC-only secondary control (blue), and cells labeled with Lin28 antibody detected by FITC secondary (red); The LIP fraction constitutes 36.2% of this live cell sample; FIG. 1C graphically illustrates a comparison of CIC detected as LIP depending upon whether cells were fixed and permeabilized versus live unfixed fresh isolate. n=4 for each condition.

### Aging Impacts Lin28 Expression.

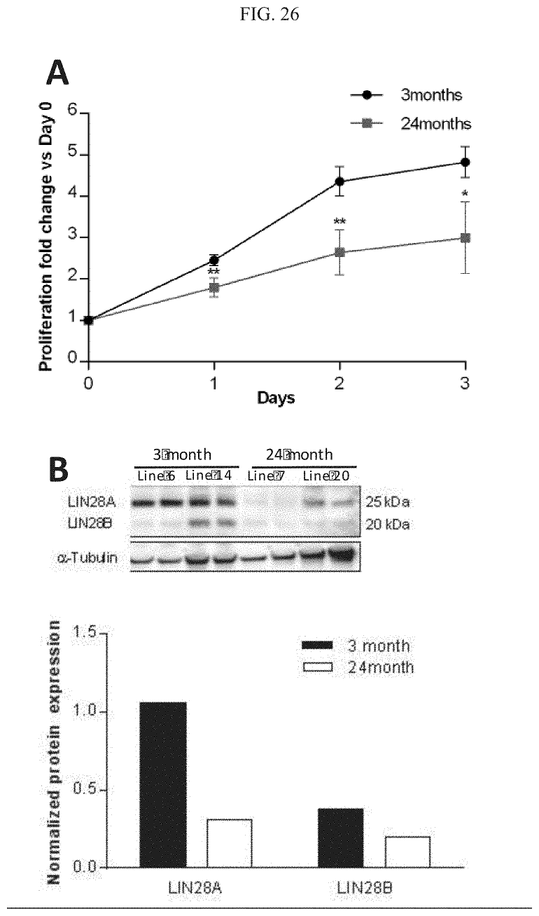

CPCs expanded in vitro from young (3 month) or aged (24 month) C57BL/6 mouse hearts were assessed for proliferation and Lin28 expression, see FIG. 26A-B. Lin28 protein level is reduced in CPCs derived from aged compared to young hearts consistent with Lin28 serving as a marker of cellular youthfulness diminished in CPCs derived from aged hearts.⁵³ Two immunoreactive bands evident in the CPCs derived from an 8 week old FVB mouse heart serve as a positive control.

FIG. 26 illustrates data showing that murine CPC aging correlates with diminished proliferation and lowered Lin28 expression: FIG. 26A graphically illustrates data showing the proliferation of CPCs isolated from young (3 month) versus aged (24 month) mouse hearts, CPCs measured using CyQuant™. 3 independent experiments, t-test versus (vs.) 3 month, significance: *=p value<0.05; **p value<0.01; FIG. 26B top panel illustrates an immunoblot of Lin28A and Lin28B, with α-tubulin as a control, and FIG. 1B lower panel graphically illustrates a quantitation of the result of the immunoblot of FIG. 26B top panel.

LIP Surface Phenotypic Profile Shows Select Differences from Other Stem Cell Populations

Low passage cultured LIP phenotype profile using hematopoietic stem cell markers is similar to that previously observed for other typical stem cell populations, as shown in Table 2:

| Markers | | | | | | | | CD117 | |
|---|---|---|---|---|---|---|---|---|---|
| Cell Type | CD29 | CD31 | CD34 | CD45 | CD73 | CD90 | CD105 | (c-Kit) | CD133 |
| LIP (Live) | 78.00% | 1.34% | 3.09% | 0.01% | 2.50% | 19.90% | 98.77% | 1.19% | 0.11% |
| CPC | 60.00% | 9.20% | 7.07% | 0.00% | 49.10% | 13.00% | 99.20% | 7.60% | 10.70% |
| CDC | 99.98% | 0.62% | 1.00% | 0.45% | ND | 18.40% | 99.89% | 7.04% | 0.99% |
| BM-MSC | 99.95% | 0.74% | 1.23% | 0.54% | ND | 99.00% | 99.37% | 5.60% | 1.24% |
| AD-MSC | 99.63% | 0.54% | 0.11% | 0.15% | ND | 84.79% | 99.68% | 10.44% | 2.17% |
| BM-MNC | 94.54% | 19.98% | 3.76% | 74.72% | ND | 4.21% | 24.54% | 3.73% | 2.17% |

In Table 2, above, the data shows the results of immunophenotyping of LIP relative to other stem cell populations including cardiosphere-derived cells (CDC), bone marrow mesenchymal stem cells (BM-MSC), adipose-derived mesenchymal stem cells (AD-MSC), and bone marrow mononuclear cell (BM-MNC). Cells assayed by flow cytometry in live unfixed state.

LIP and CPC percentiles shown in bold font were obtained from our group with other values shown from a previous publication.⁵⁴ Notably LIP expresses low levels for select EPC (CD133) and MSC (CD73) markers compared to other stem cell types while exhibiting levels of other markers analogous to previously described stem cell populations (CD29, CD31, CD34, CD90, CD105, CD117). LIP as well as CPC are negative for the hematopoietic marker CD34. Thus, LIP immunophenotypes as CICs that diverge from previously characterized populations in at least two surface markers consistent with the postulate that LIPs represent a differentially selected subset of CICs.

LIP Paracrine Factor mRNA Levels Markedly Increased.

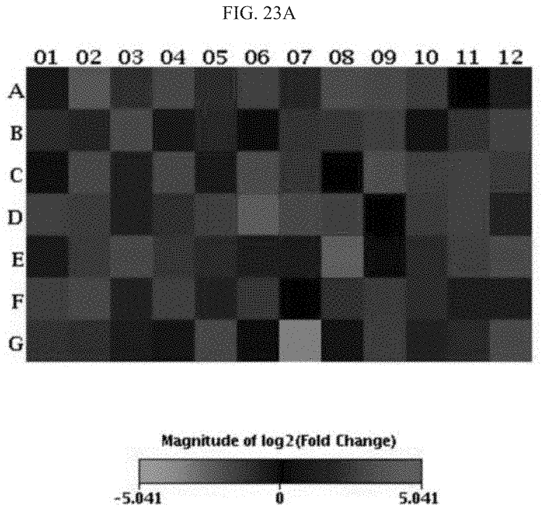

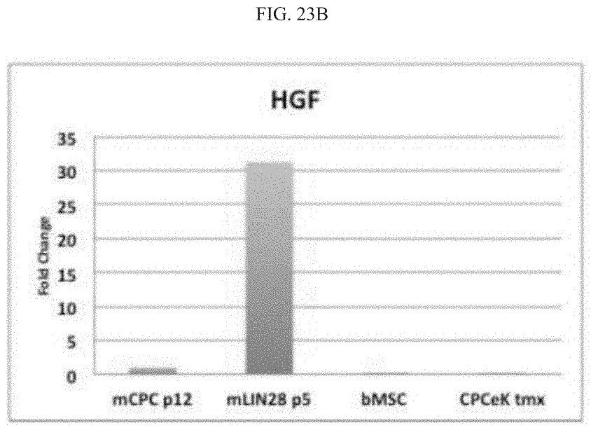

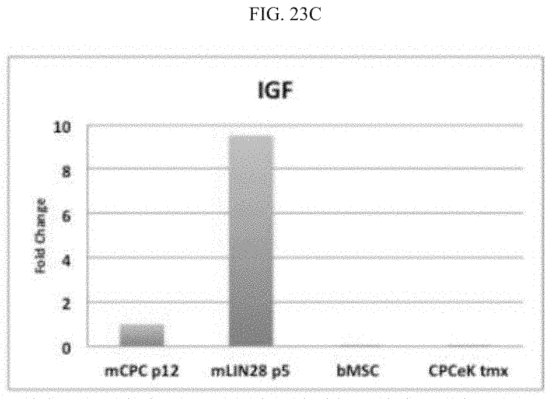

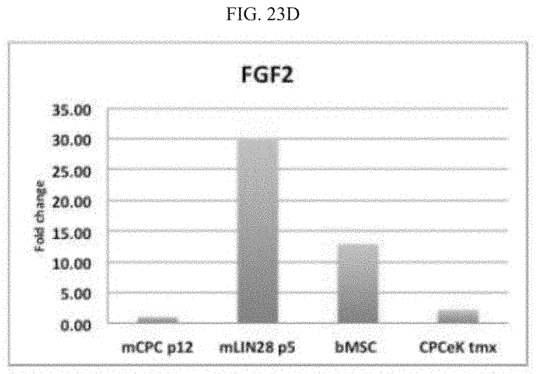

The secretome is touted to be responsible for beneficial effects of adoptively transferred stem cells.⁵⁴⁻⁵⁸ Assessment of paracrine factor mRNA levels by commercially available qPCR assay plates (SAB Biosciences) revealed dramatic and significant increases in HGF, IGF, and FGF2 mRNAs in LIP relative to CPC or MSC confirmed by primer-specific qPCR, as graphically presented in FIG. 23B-D. Dramatic elevations in mRNAs of paracrine factors known to exert profound cardioprotective and trophic effects upon stem cells support the biological action of LIP in mediating myocardial response to injury and potentially accelerating endogenous cell recruitment, survival, and proliferation.

### LIPs Exhibit Upregulated RNA Expression of Paracrine Factors

Adult mouse LIP at passage 5 (mLIN28 p5) display higher expression for HGF (FIG. 23B), IGF (FIG. 23C), and FGF2 (FIG. 23D) compared to mouse CPCs passage 12 (mCPC p12), bone marrow-derived MSC (bMSC), or mouse CPC genetically modified to express intracellular Notch domain (CPCeK tmx) by qPCR. Ribosomal RNA signal used for sample normalization. Relative fold change indicated on Y-axis.

LIP Morphometry, Dynamics, and Nucleation In Vitro are Unique and Distinct from Well-Known Adult Cardiac Stem Cells

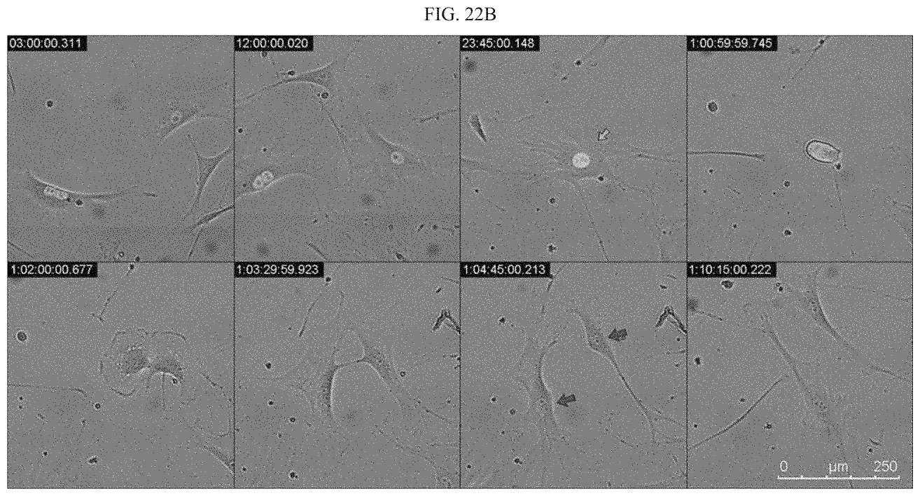

Characteristics that distinguish LIP from CPC in vitro were evident from observing expanded cultured cells by time-lapse video microscopy, see FIG. 22A-B, illustrating LIP cell dynamics revealed by time-lapse video microscopy; two series of still frames of LIP cells with time stamp (upper left of each panel); the upper series of eight panels show a cell expressing FUCCI reporter entering mitosis as binucleated (green arrow, upper row third frame) indicated by green nuclear fluorescence reporter and after exit from mitosis showing red fluorescence giving rise to two mononucleated daughter cells; the lower series of eight panels shows a mononucleated cell (green arrow) giving rise to two mononucleated daughter cells (red arrows); scale bar=250 μM.

Readily apparent differences present in the LIP were the large size of many cells (over 150 mM in length) as well as frequent presence of binucleated cells that consistently undergo mitotic division resulting in two mononuclear daughter cells. LIP cells were modified by lentiviral infection to express FUCCI fluorescent cell cycle regulator to confirm progression through mitosis (shift from red to green nuclear fluorescence).⁵⁹⁻⁶¹ These characteristics distinguish LIP from in vitro phenotypic properties of CPC, MSC, or EPC as reported by our group⁶² and highlight unexpected binucleated cell mitotic activity in LIPs.

### Selective Enrichment of LIP Produces Distinct Subpopulations.

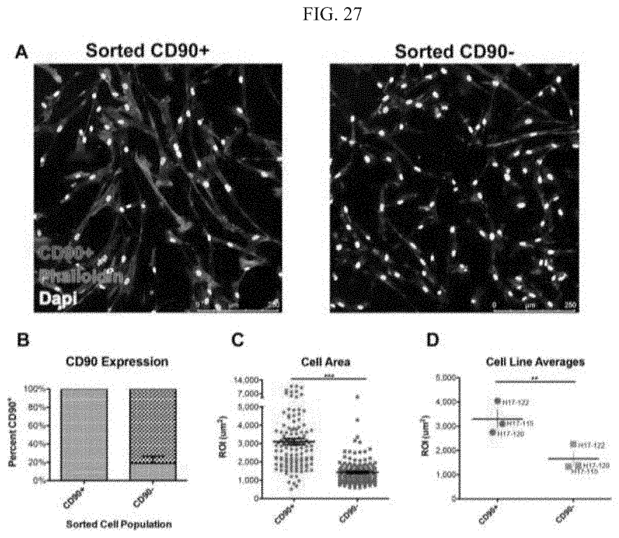

Depletion of CD90+ cells enhances reparative potential of cardiosphere-derived cells (CDC) in a rodent myocardial infarction model, although characteristics of the CD90 depleted subpopulation were not defined.⁴⁵ Immunoselection of LIPs using CD90 antibody yields significantly distinct cell populations as defined by morphometric analyses, see FIG. 27A-D. Interestingly, the CD90+ population consists of larger cells consistent with the postulate that this population represents functionally distinct interstitial cells. These findings were reproducible with LIPs isolated and phenotyped from three different human heart failure samples (FIG. 27D). These initial findings validate LIP heterogeneity and the conceptual approach of using subsequent selection to further enrich for LIP subsets possessing biologically and functionally distinct properties.

FIG. 27A-D illustrates LIP CD90 immunoselection yields distinct subpopulations. LIP subset properties revealed by confocal microscopy (FIG. 27A), CD90 expression (FIG. 27B), area (FIG. 27C) and average area for three human LIPs (FIG. 27D); Significance: ***=p<0.001; **=p<0.01.

### Transcriptome Comparison Reveals Distinctions Between LIPs Versus CPCs.

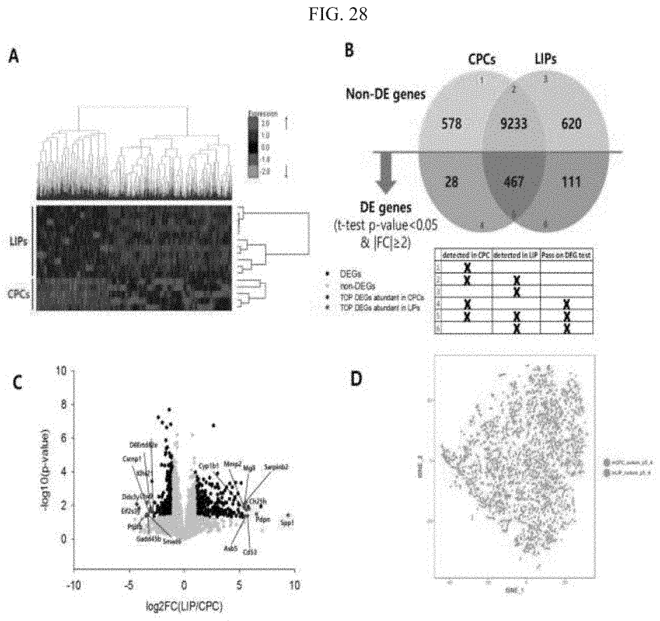

Biological distinction between LIP versus CPC was confirmed by single cell RNA-Seq analysis performed on individual cells (n=6 for each group) using Smart-Seq cDNA library creation followed by Illumina sequencing (7.1 million reads per cell) and bioinformatics analysis using BaseSpace™ software (Illumina) all performed by our group. Divergence between LIP versus CPC cells at the transcriptome level was evident from single cell RNA-Seq results, see FIG. 28A-D. Specifically, 606 differentially expressed genes corresponding to 5.5% of the transcriptome were detected by comparing LIP to CPCs. Contextualizing this result, genes corresponding to a single lineage-enriched cardiovascular cell type comprise only 1.43 to about 3.19% of all detected genes, whereas 80˜84% of genes are expressed at similar levels throughout cardiovascular development.⁶³ Clues as to the biological nature of the LIP population relative to CPCs were furthered by massively parallel single cell RNA-Seq performed on human cultured cell populations using the 10× Genomics Chromium platform recently acquired by our group. A total of 1,850 cells with mean averages of 267,620 reads per cell and 5,480 detected genes confirmed population transcriptional heterogeneity and reveals CPCs as a closely related subpopulation of LIPs (FIG. 28D).

FIG. 28A-D illustrates single cell RNA-Seq comparative analysis of LIP versus CPC: FIG. 28A illustrates a heatmap showing differentially expressed genes between LIP and CPC single cells (n=6 cells for each population, 7 million reads per cell); FIG. 28B illustrates a Venn diagram representation of differentially expressed (DE) genes (defined as fold change>2 and p value<0.05). From a total of 606 DE genes, 28 are unique to CPC, 111 are unique to LIP, and 467 are shared. Legend below diagram shows the various subgroups of the comparative analysis; FIG. 28C illustrates a Volcano plot representing the top nine DE genes between CPC and LIP populations with most abundant CPC DE genes labeled in blue versus most abundant LIP DE genes in red. Remaining DE genes (black dots) and non-DE genes (gray dots) are shown; FIG. 28D illustrates an tSNE population analysis demonstrating relationship between LIP (turquoise) versus CPC (salmon) populations.

### Dramatic Expansion of LIPs in Response to Infarction Injury.

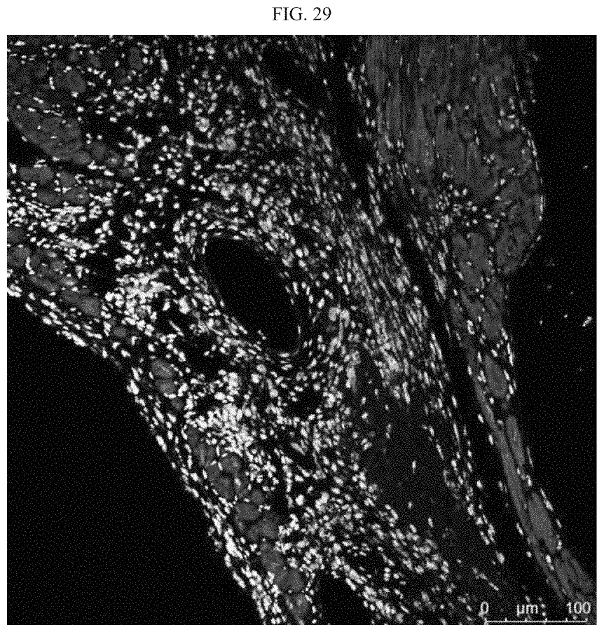

LIP distribution shifts markedly following infarction injury with proliferation and migration into the infarct as well as border zones, see FIG. 29. LIP infiltrates are widespread and highly dynamic, changing in both density and localization throughout the course of post-MI remodeling well after inflammatory responses and CD45+ cells of hematopoietic origin have dissipated (data not shown). LIPs far outnumber CPCs in the CIC following injury, suggesting these cells likely play a significant role in myocardial repair, scar formation, and long term structural and functional outcome.

FIG. 29 illustrates an image of LIP expansion following infarction injury: a confocal scan of mouse heart paraffin sections at 7 days post-MI immunolabeled for Lin28 (green), c-Kit (red), cardiac troponin T (cTnT, blue) and DAPI (grey). LIPs appear in the remodeling zone of infarction damage and in proximity to, but distinct from, cardiac c-Kit cells.

### LIP Exhibits Profile of Stemness Relative to Lin28− CICs.

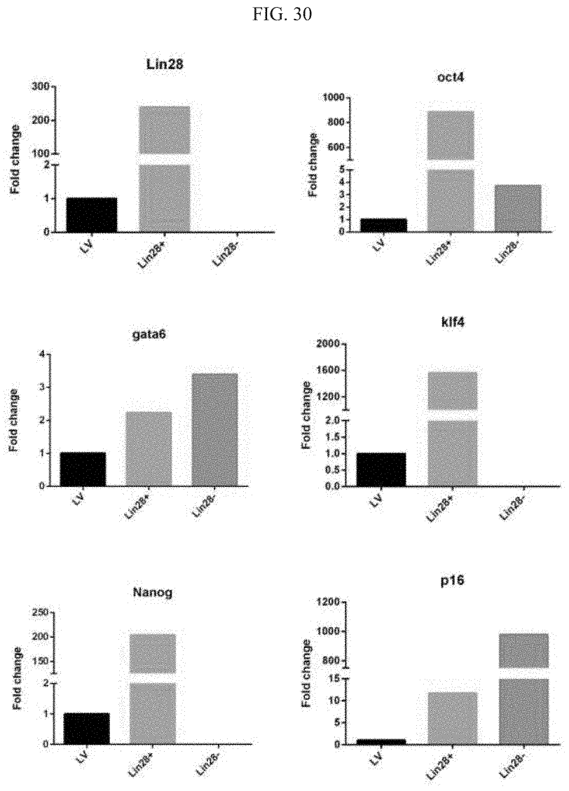

Profiling of fresh murine isolates confirms the presence of elevated stemness markers Lin28, Oct4, KLF4, and Nanog in LIPs relative to Lin28− CICs, see FIG. 30. Interestingly, the senescence marker p16 is elevated in Lin28− CICs. The cardiogenic marker Gata6 is not significantly different between the two cell populations. Collectively, these initial findings support the premise that Lin28 is a selectable marker to define a novel subset of relatively “youthful” CICs (a.k.a. LIP).

FIG. 30 graphically illustrates data showing elevated stemness markers in LIPs. mRNA levels for selected markers of stem cells (Lin28, Oct4, Klf4, Nanog), cardiovascular (Gata6), or senescence (p16). Performed using validated primer sets on fresh murine LIP or Lin28− CIC normalized to total left ventricular mRNA (LV) as well as loading variations.

### LIP as a Novel Population for Mediating Myocardial Homeostasis, Repair, and Regeneration

As a CIC subpopulation, characterization of LIPs reveals a heterogenous stem cell pool expected to possess biological properties of pluripotent “youthful” cells consistent with phenotypic traits previously ascribed to Lin28 expression (FIG. 30).^{3, 8, 11, 28-30, 43} Two important aspects of understanding LIP biology are 1) determining how this endogenous population changes over lifespan and adapts to pathological damage, and 2) using Lin28 as a selection marker to optimize selection of CSC for initiation and promotion of myocardial repair and regeneration. Conceptually, using a marker of cellular pluripotentiality such as Lin28 advances the premise that biological functional efficiency rather than cell type is a critical determining factor in outcome of myocardial healing. Although Results indicate LIPs are phenotypically and functionally distinct from CPCs and share some properties with MSC, it is paramount to envision LIPs as heterogeneous population that can be further optimized by subsequent (follow-up) selective steps (see FIG. 27A-D). With respect to heterogeneity, LIPs share this characteristic with CDC, but important distinctions include: 1) no marker specifically defines CDCs and it cannot be studied as an “endogenous” cell population, 2) CDC can never be isolated as a fresh population as their existence is defined by outgrowth in cell culture that changes their biology, 3) CDC are not selected based upon a biological properties of marker expression or biological potential. In contrast, use of Lin28 implies that selection and expansion of LIPs provides cells with enhanced functional competency.

Collecting LIPs Directly from Human Heart Tissue Samples by Affinity Bead Enrichment, Performing Assessments on Freshly Isolated Cells for Molecular, Biochemical, and Biological Properties

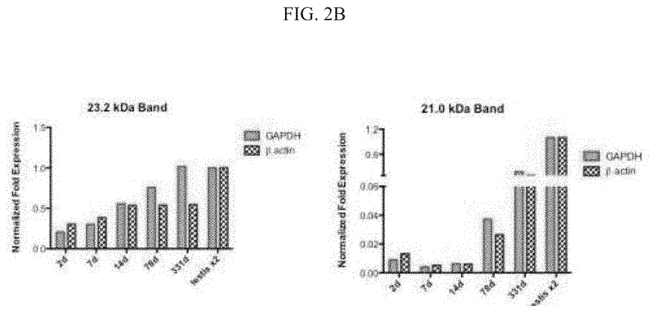

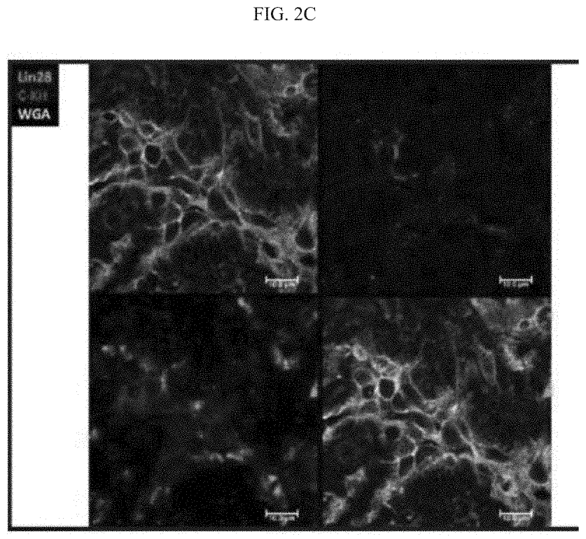

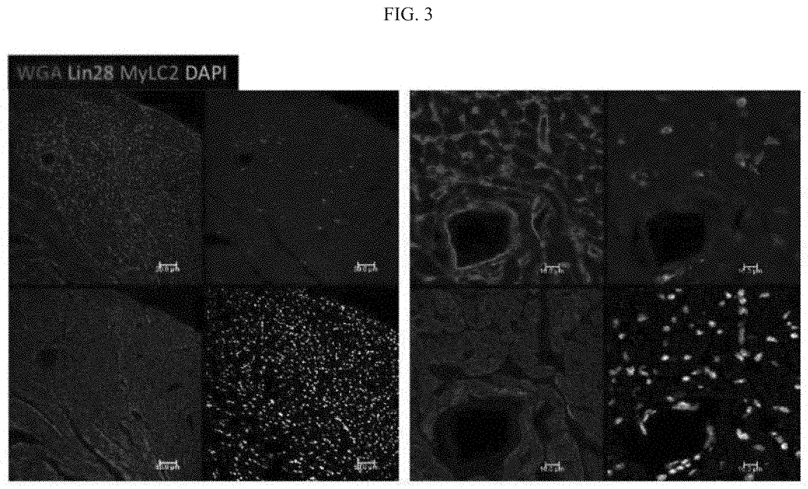

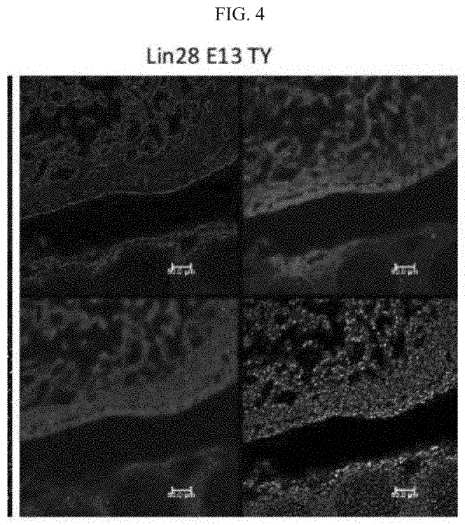

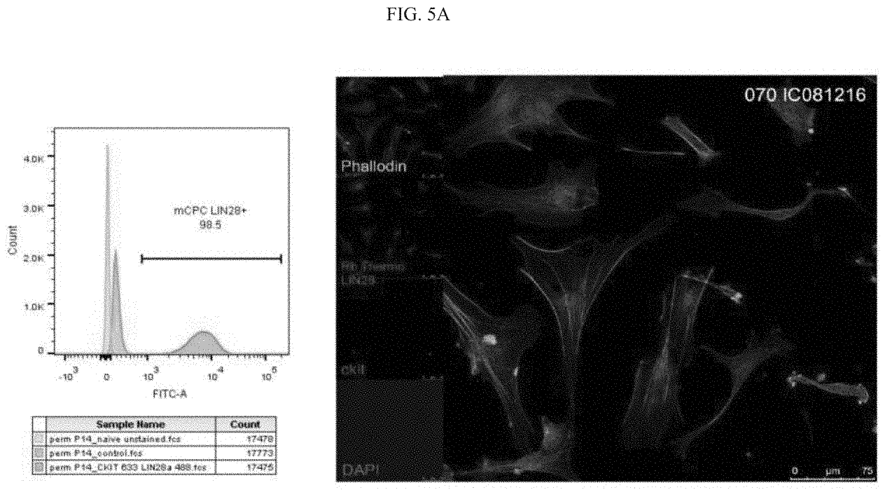

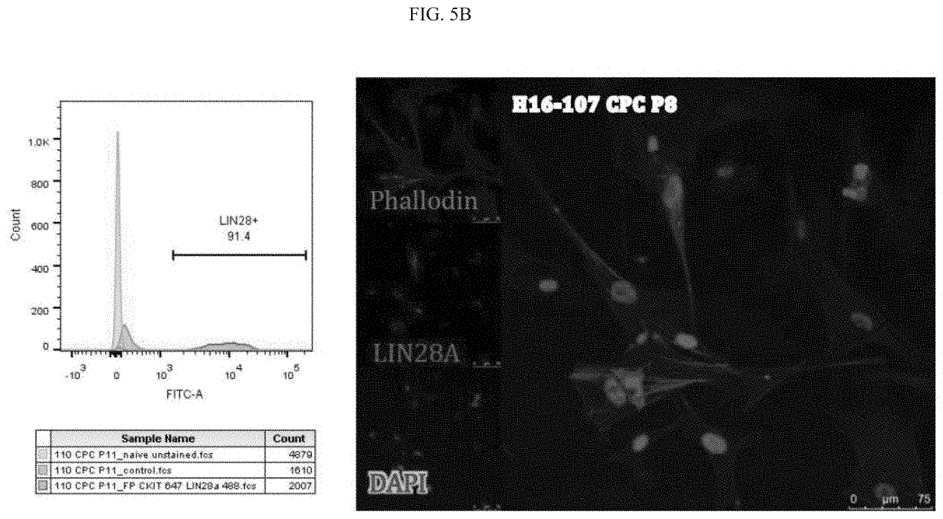

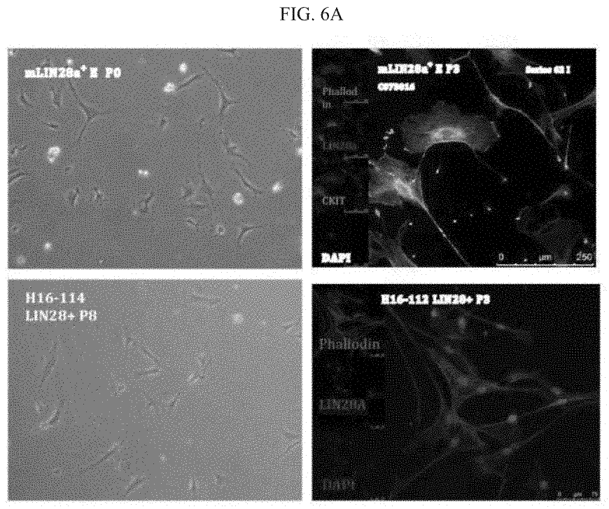

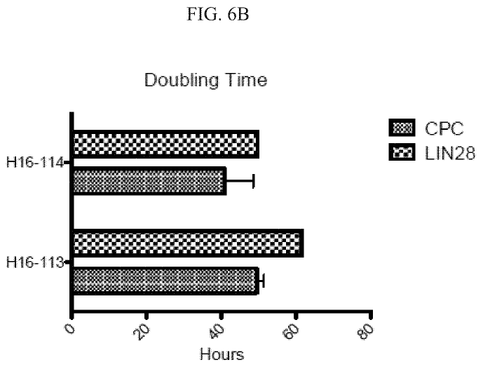

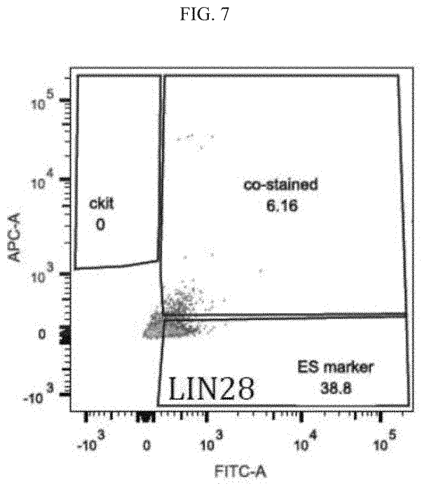

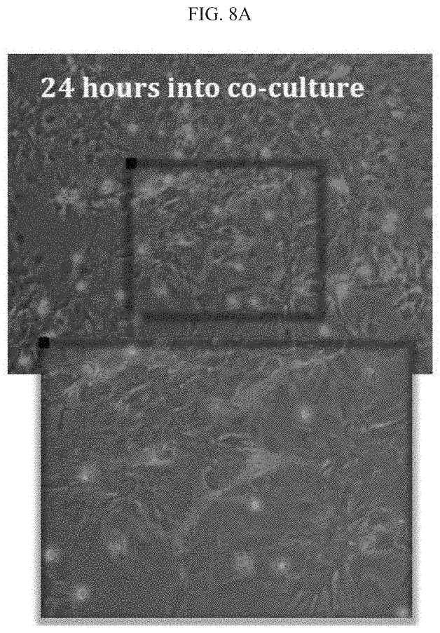

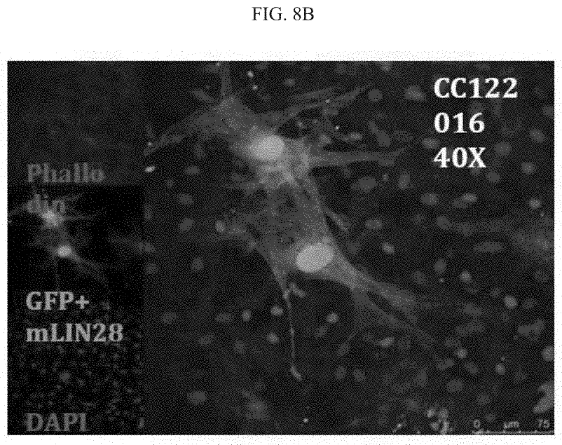

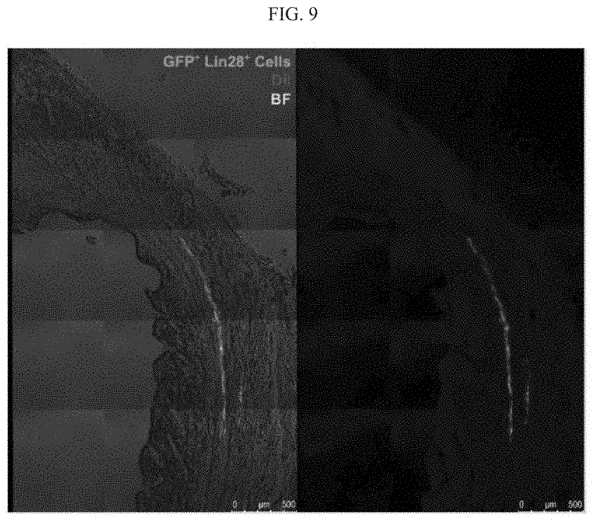

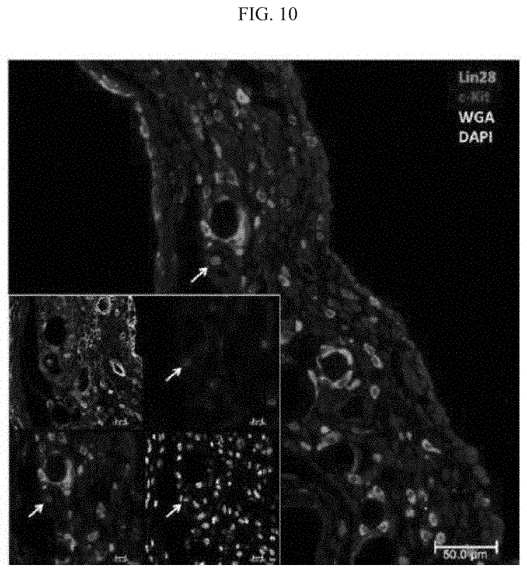

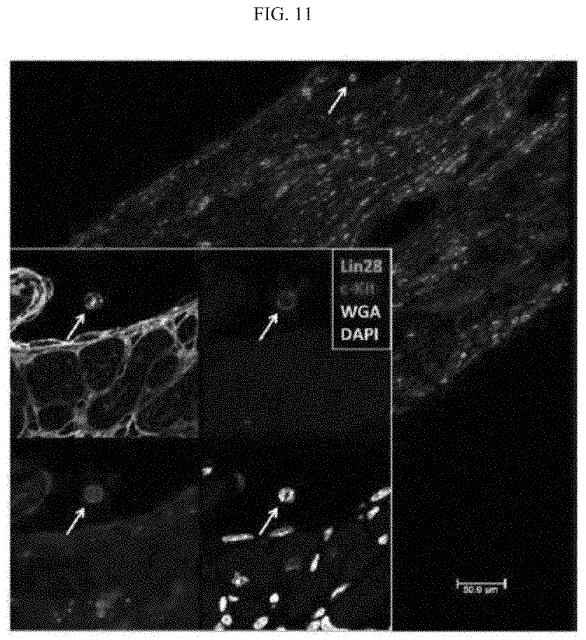

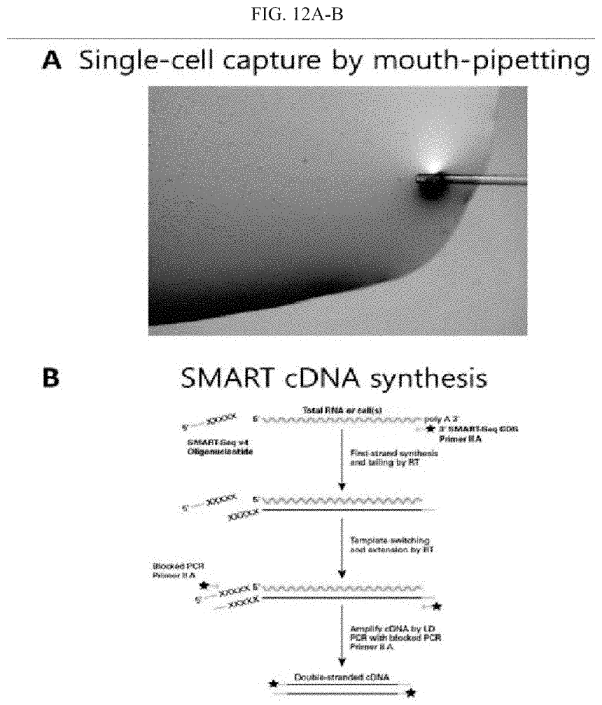

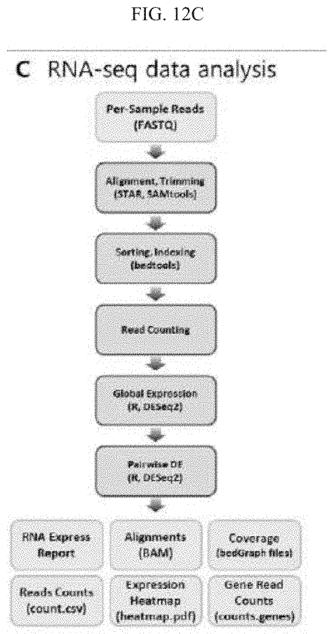

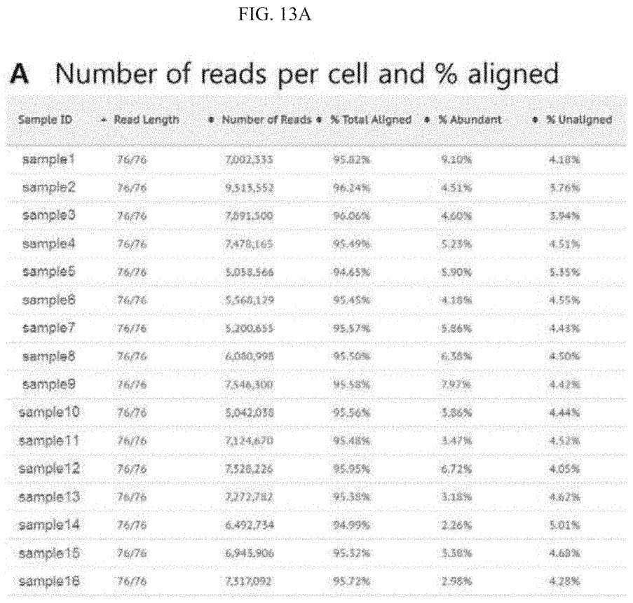

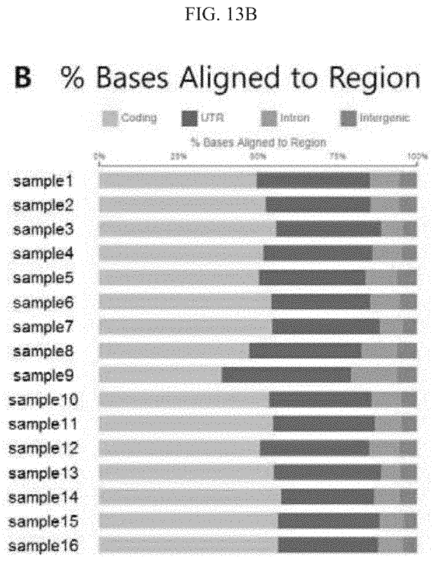

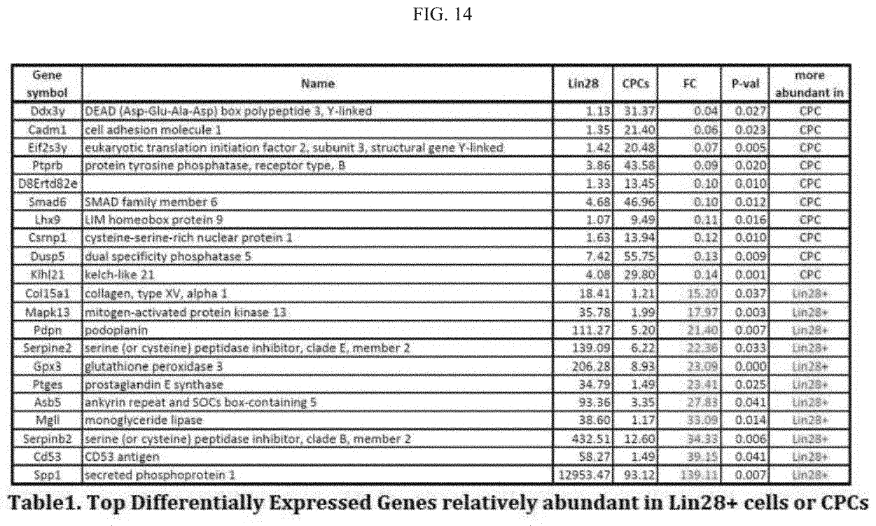

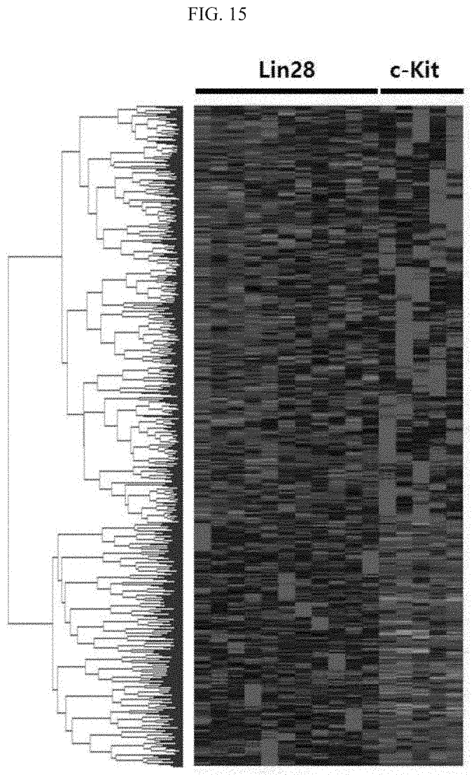

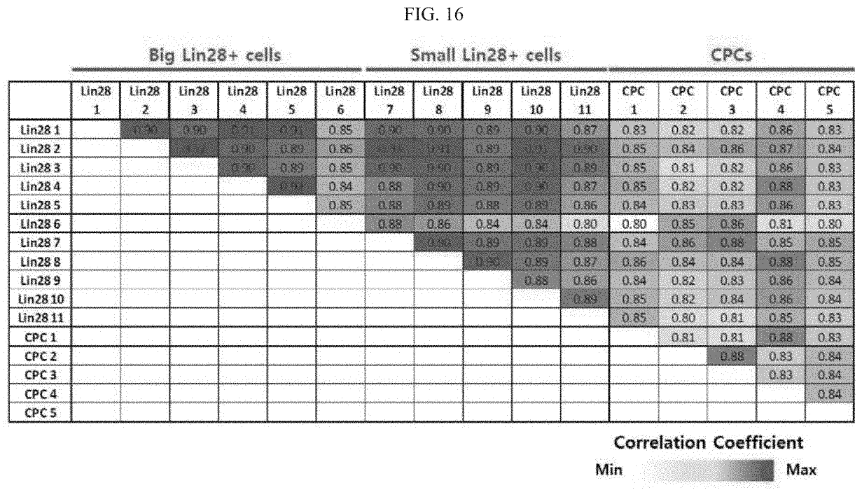

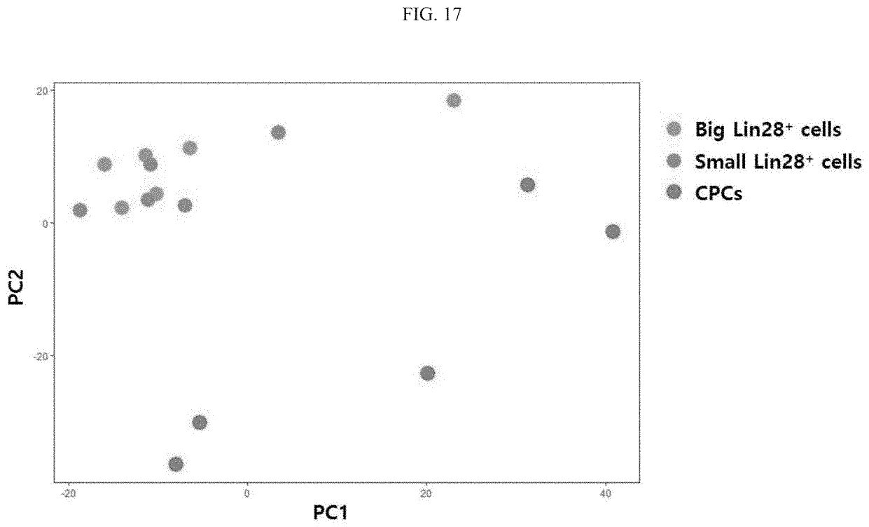

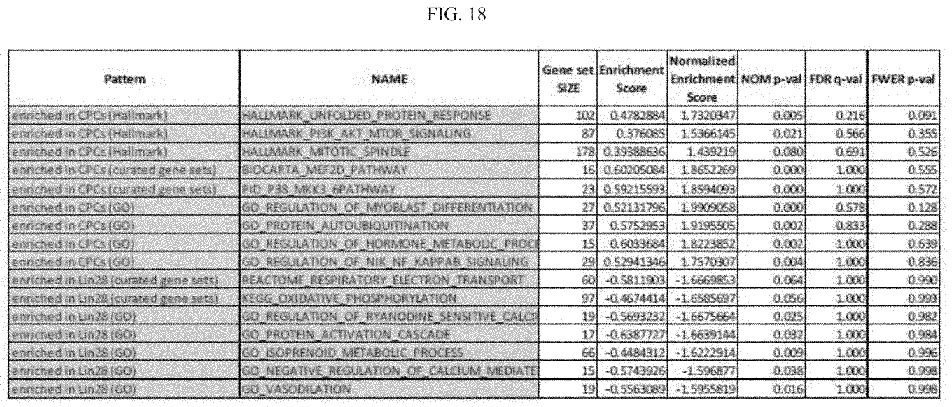

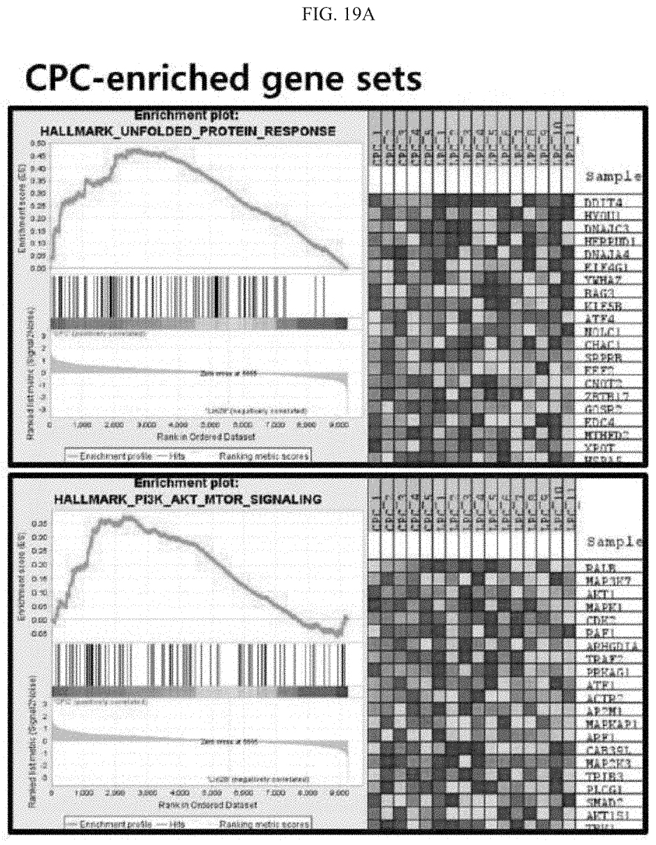

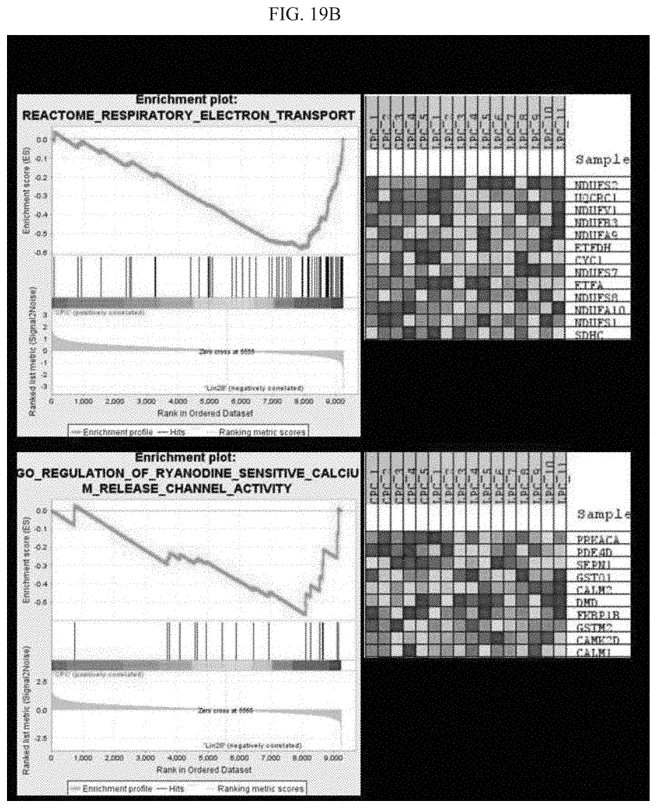

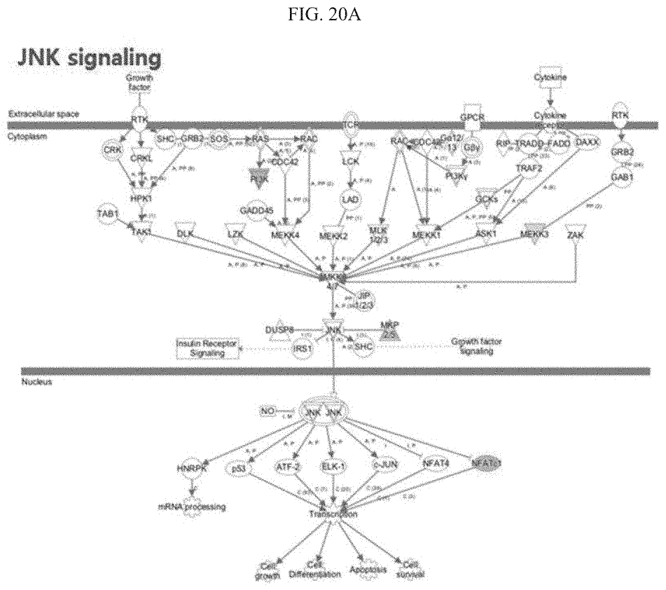

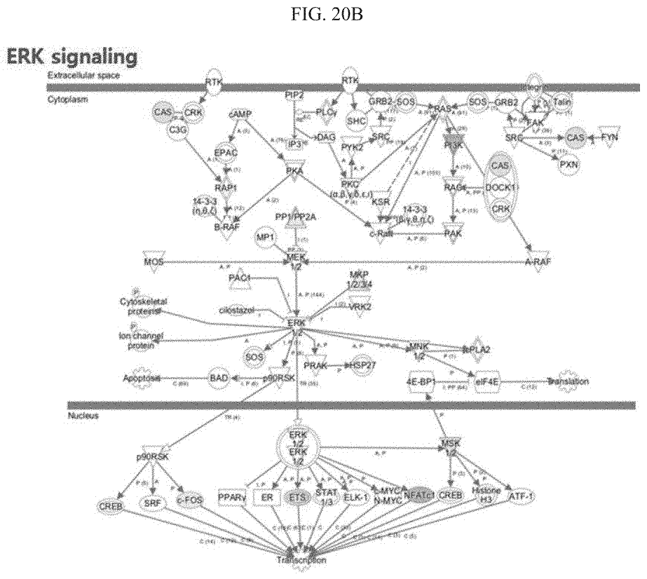

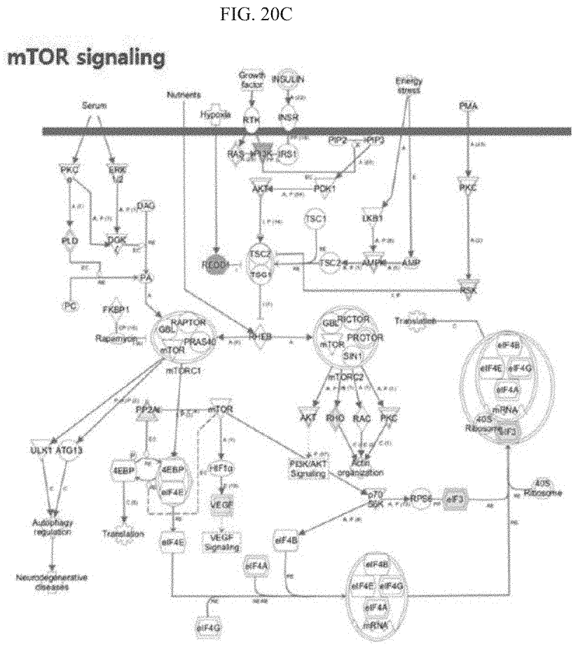

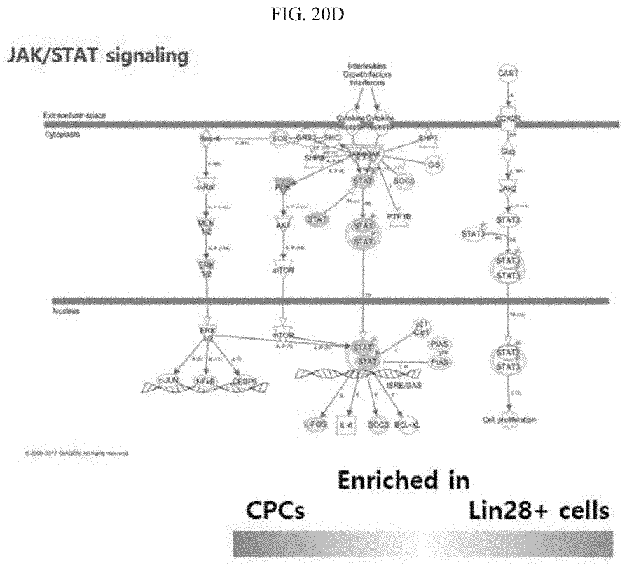

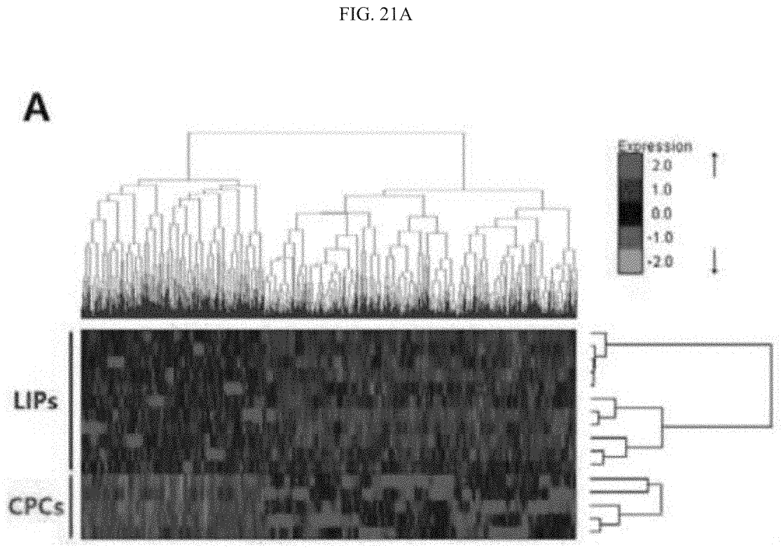

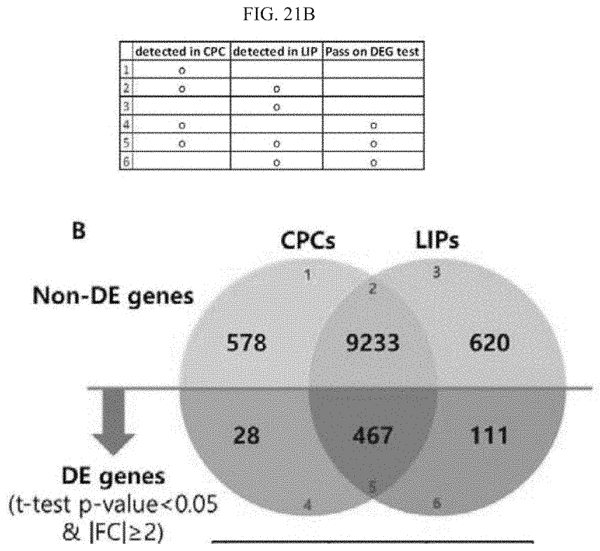

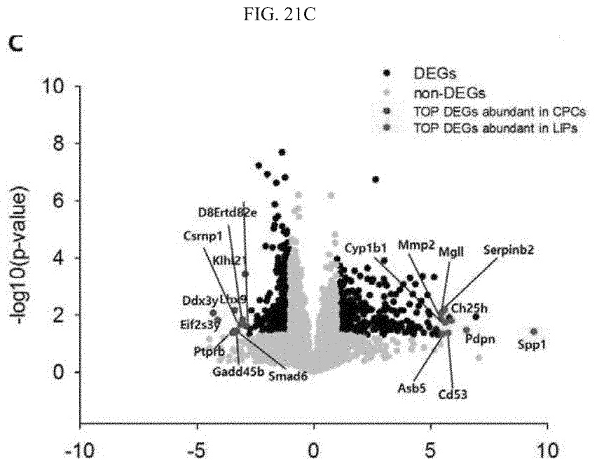

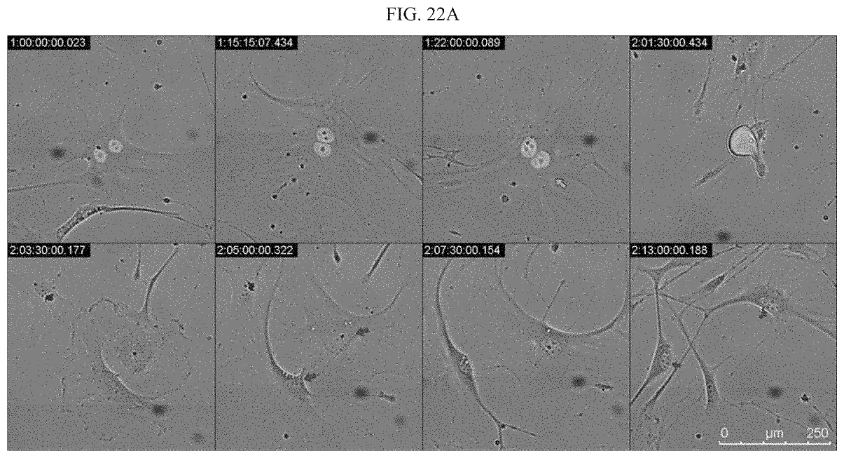

Fresh human heart tissue obtained from LVAD recipients consists of a “plug” removed from the left ventricular free wall at the time of pump insertion. The tissue is placed into ice cold cardioplegic buffer and transported directly to our lab for further processing. Additional methodologic details are similar to those published by our group for isolation of other cardiac stem cell types from the nonmyocyte fraction of human heart tissue samples.⁶² LIP enrichment is performed using Lin28 antibody (Thermo-Fisher PA1-096) in combination with MACS bead affinity selection to enrich for LIP, whereas cell flow through of the column is collected as Lin28− CICs. Selected cell fractions are expanded in vitro to produce sufficient numbers for archival frozen storage as well as use in experiments within 3-4 sequential splits from the stage of initial isolation. Typical yield from an LVAD sample produces 15,000-21,000 CICs to be processed by MACS immunoaffinity selection, yielding sufficient amounts for experimental studies in this Aim. LVAD tissue samples are available 3-5 times per month based upon our experience over the last several years, so we can readily produce cell preparations from a dozen patients within a few months. Characterizations can focus upon basic differences between Lin28+(LIP) versus Lin28− CIC fractions. Our protocol also allows for simultaneous isolation of c-kit+ CPCs in tandem with LIPs from the same patient sample, allowing determination of the relationship between LIPs and CPCs derived from one individual to understand what LIP properties may be superior to that of traditional CPC preparations. The high yield of LIP and Lin− CIC populations from myocardial tissue (FIG. 2) enables direct assessments of freshly isolated cells for biological and functional parameters. Comparative analyses between LIP versus Lin28− CIC characteristics can be established for susceptibility to apoptotic challenge, morphometrics, secretory profiling, senescence-associated signaling, phenotype and markers by flow cytometry, telomere length, cell cycle regulation or arrest molecular signaling, karyotyping, and motility/mitosis revealed by live cell time-lapse video microscopy all as previously published by our group^{49-51, 62, 64-67} Concomitantly, single cell transcriptional profiling can be performed by RNA-Seq using the 10× Genomics CHROMIUM™ platform and bioinformatics capabilities recently acquired by our group (FIG. 7; manuscript in preparation).

### Performing Confocal Analyses on Human Tissue Sections to Delineate LIP Biology In Situ.

Histologic analyses can be performed using human tissue samples derived from human failing heart samples obtained for cell isolation as noted above. In addition, samples of fixed human heart for have been obtained from the National Disease Research Interchange (NDRI; http://ndriresource.org) and Cooperative Human Tissue Network (CHTN; https://www.chtn.org) including both paraffin-embedded tissue blocks for microscopy as well as snap-frozen tissue chunks. Lin28 immunolocalization can be studied in samples from human adult mid-life non-pathologic heart tissue of both male and female origin with varying ethnicity to seek potential variations between samples. Location, frequency, and intensity of Lin28 immunolabeling can be assessed as well as colocalization with markers for CSC (see Table 2 and ¹) as well as CIC (e.g. vimentin, Tcf21, PDGFR, FSP, etc. as delineated in⁶⁸⁻⁷⁰).

### Validate Human Findings in the Murine Context to Establish an Experimental Animal Model.

Having established biological and functional parameters of human LIPs earlier in this aim, additional studies can correlate findings between human versus murine LIPs. These studies can provide clarification of mouse LIPs as a suitable experimental platform to study biological processes that advance insights toward translational application. Molecular, biochemical, and biological properties of murine LIPs can be established using approaches and analyses comparable to those for human LIPs previously described herein. Attention can be focused upon significant differences between mouse versus human LIPs, as these may provide valuable clues regarding divergence in biology between human versus mouse myocardial interstitial cells. Typical mouse laboratory strains of FVB and C57BL/6 including both male and females at mid-life (6 months) can be examined to evaluate if strain or gender-specific differences in LIPs are present.

Lin28 can reveal a distinct stem cell population in LIPs characterized by “youthful” characteristics including enhanced resistance to apoptosis, smaller size, greater secretory activity, decreased senescence characteristics, concurrent markers with a heterogeneous group of CICs including stromal cells, longer telomeres, high cell cycle rate (˜24 hour doubling time) and expression of cycle markers, normal karyotype, and high motility rates all relative to the Lin28− CIC population. Single cell transcriptomics can reveal LIP heterogeneity, differentially expressed genes associated with pluripotency, and rare populations with distinct transcriptional profiles with potentially enhanced functional characteristics relative to Lin28− populations, Confocal analyses can demonstrate interstitial distribution of LIP as well as coincidence with specific markers for previously characterized interstitial cells types, reinforcing the heterogeneous nature of LIP phenotypic traits. Parallel studies between human and murine samples for these properties are anticipated to demonstrate comparability between the species with respect to distribution, colocalizations, functional, phenotypic, transcriptomic, and chronologic features. Murine LIPs may exhibit some blunted aspects related to aging phenotypes relative to humans due to shorter lifespan as well as unusually long telomeres in mice, so traits such as markers of cell cycle arrest and senescence (including DNA damage) can be emphasized.

Lin28 Expression with Aging Demonstrated by Immunoblot Analysis.

A biobank of human heart tissue lysates collected from individuals ranging in age from fetal development to over 80 years old has been curated over several years as previously published⁷² and pediatric samples can be used. Specific ages can be determined pending sample availability, a range from fetal to old age can be used using nonfailing myocardial samples inclusive of both genders. Immunoblot findings can be corroborated from the murine studies detailed above with a range of human heart samples intended to demonstrate the expression of Lin28 throughout lifespan. Similarly, Lin28 expression in adult myocardium is predominantly associated with LIP CIC immunoreactivity. This observation can be further developed by immunoblot analyses of murine CIC versus myocyte fraction to validate Lin28 expression levels in myocardial tissue. Once having determined the relative distribution of Lin28 in the CIC versus myocyte fractions, subsequent (follow-up) analyses can focus upon quantitative immunoblot determinations of Lin28 protein expression in CIC (and myocytes if present) throughout postnatal development through old age in murine cardiac samples. Straightforward analyses of murine tissue lysates of both male and female origin can be performed at time points of postnatal days 2, 5, 10, 14, and 21 to capture myocardial transitions from early mitotic growth to later hypertrophic remodeling. Additional samples at 1, 2, 6, 9, 12, 18, and 24 months can be similarly assessed to determine levels of Lin28 expression over the lifespan, with samples from both FVB and C57BL/6 mice to be used to assess variation between strains.

### Changes in LIP Distribution and Frequency Over Lifespan Revealed by Confocal Microscopy.

Histologic analyses can be performed using human tissue samples derived from human failing heart samples obtained for cell isolation as noted above. In addition, samples of fixed human heart have been obtained from the National Disease Research Interchange (NDRI; http://ndriresource.org) and Cooperative Human Tissue Network (CHTN; https://www.chtn.org) including both paraffin-embedded tissue blocks for microscopy (see FIG. 1 right panel) as well as snap-frozen tissue chunks. Samples can vary in age and ethnicity as determined by sample availability, and cover a full spectrum from pediatric, adolescent, mid-life, to aged tissue. Pursuing a strategy comparable to that described for immunoblot analyses in the preceding paragraph, samples from mice at multiple time points throughout life can be prepared for confocal microscopy analyses utilizing protocols developed for assessment of Lin28. Immunolabeling for Lin28 can be performed in conjunction with colocalization for myocyte, endothelial, vascular, and interstitial cells including other landmarks such as nuclei or sarcolemmal membranes as previously published by our group.^{49-51, 62, 64-67, 72, 73} Tissues can be prepared by paraffin embedding to preserve architectural information and coincidence of Lin28 labeling evaluated by optical sectioning and 3D stack reconstruction as appropriate to confirm observations.

LIP Characteristics Compared Between Pathologically Damaged Myocardial Samples from Human as Well as Murine Experimental Models of Infarction and Hypertrophy.

Fundamental information regarding Lin28 expression and localization throughout lifespan earlier in this aim can guide subsequent (follow-up) studies in this subsection to determine the impact of myocardial pathogenesis and disease upon Lin28 expression and LIP distribution. Pathologic human samples can be analyzed from various sources including the National Disease Research Interchange (NDRI; http://ndriresource.org), Cooperative Human Tissue Network (CHTN; https://www.chtn.org), and samples provided by collaborating cardiac surgeons (see support letters). Varying etiologies can be assessed including chronic heart failure, myocardial infarction, chronic hypertension leading to failure, congenital cardiomyopathies, and genetic-based hypertrophic remodeling. Sample distribution between age, gender, and ethnicity are unbiased and inclusive. Assays can include confocal microscopy, immunoblotting, and MACS-bead immunoaffinity isolation to assess fresh (non-cultured) LIP versus Lin28− CIC cell preparations when harvested human samples are available for single cell transcriptomics. Murine samples can be derived from typical acute injury models as previously studied by our group including infarction, hypertrophy, and dilated cardiomyopathy.^{66, 67, 74-78} Time points proximal to injury can be assessed for LIP characteristics by the same aforementioned criteria enumerated for human samples. Infarction and hypertrophy injury can be assessed relative to sham at post-surgery days 3, 7, 14, 21, and 45 days, whereas dilated cardiomyopathic samples can be assessed relative to normal mice at 6-8 weeks of age.

Lin28 expression correlates with chronologic age and decrease over lifespan. However, there may be reservoirs of LIPs that retain high Lin28 expression well into advanced age and such cells could represent “immortal” CICs with preserved functional properties in old myocardium. Findings show impressive expansion of the LIP at 7 days post-MI in mice, so it is reasonable to anticipate that LIPs can respond acutely to pathologic injury with increased cell numbers ad wide distribution throughout the injured area. The arc of accumulation and disappearance of LIPs with MI is expected to occur over a period of weeks that peaks between 7-14 days after injury. In hypertrophic or chronic stress conditions, LIP distribution can likely be widespread, uniform, and increased overall relative to non-injured age and gender-matched samples. The combinatorial approach of quantitative immunoblotting together with confocal immunolocalization can provide mutually reinforcing but distinct characteristics of LIP myocardial biology. Transcriptomics can reveal drift and changes in the profile of LIP heterogeneity, as subpopulations with distinct transcriptional signatures are expected to rise up and fall over the time course under analysis, providing important novel information on population dynamics of LIPs during pathogenic remodeling.

Derivation of LIP Cultures Resulting from In Vitro Expansion Amplifies a Subpopulation with Phenotypic Characteristics Influenced by Passage Number and Co-Culture Environment

A primary requirement for most cell therapy approaches is the expansion of initial isolates (either autologous or allogeneic) to sufficient quantity for adoptive transfer into the recipient. However, considerations of cellular phenotypic drift with expansion passaging are rarely, if ever, addressed. Studies from our group documented that initial heterogeneity of freshly isolated primary cell cultures progressively and quickly narrowed with subsequent adaptation of cells to their new in vitro environment. With respect to LIPs, ongoing higher order passaging leads toward expansion of cells exhibiting smaller surface area and reduction in the number of large cells showing flattened morphology with multinucleation and alterations of the transcriptome. This selection process, likely a Darwinian pressure for better adapted cells, is likely to have significant consequences for LIP biological properties that could be critical for therapeutic value. Defining evolution of LIPs in culture can be essential to choose the right cells for adoptive transfer experiments, and can also reveal an important yet relatively unexplored aspect of cardiac stem cell biology with clear implications for therapeutic advancement.

### Expand Human LIP In Vitro, Harvest Low (<10) Versus High (>20) Passage Cultures for Analysis/Comparison Relative to Each Other or Lin28− CIC.

LIP and Lin28− CIC cultures can be maintained under normal culture conditions. Fresh (non-cultured) LIP versus LIP expansion in vitro can be performed to generate samples at passages 5 versus 22 post-isolation. Passage points were chosen to reflect early versus late LIP accommodation to culture conditions. Comparable preparations can be prepared for Lin28− CICs for alternative cardiac cell population controls.

### Massively Parallel Single Cell Transcriptome Analysis.

Massively parallel single cell transcriptomics has arrived as a platform for cell/molecular biology laboratories⁷⁹, including e.g., a 10× Genomics Chromium™ platform to analyze scRNA-Seq of various CIC populations. The 10× Chromium™ controller pipeline offers the desirable combination of high cell throughput, efficient cell capture rates, user-friendly protocols, all required equipment and reagents, as well as robust post-sequencing bioinformatics software applications.

scRNA-Seq of LIP and Lin28− CIC populations can be performed to understand their inherent biological properties and heterogeneity, comparing fresh isolates to cultured cells to define adaptations and alterations resulting from natural selection pressure in vitro. Trial runs led us to settle upon profiling between 1,000-2,000 cells per run at a read depth of approximately 70,000-90,000 per cell. For transcriptome dataset visualization and analysis, Loupe™ Cell Browser is a desktop application that allows quick and easy visualization and analysis of 10× Chromium™ Single-Cell 3′ data, optimized for finding significant genes, identifying cell types, and exploring substructure within cell clusters. Once displayed in Loupe™, tSNE plots can be interrogated for gene expression using Library IDs, subsets selected by lasso function, split of overlay view, and clustering rendered though graph-based/K-Means analysis. Embedded features allow for generation of heatmaps, DEG lists, snapshots of outputs, and gene description direct links to Ensembl™ genome browser (https://www.ensembl.org/index.html).

### Dynamics of LIP Morphometric Changes and Nucleation State Revealed Using Video Microscopy.

Time-lapse video microscopy has revealed unexpected and provocative features of LIPs in terms of morphology, mitotic activity, and dynamics in culture conditions. Capture of LIP versus Lin28− CIC cultured cells at the two proscribed adaptation points of 5 and 22 passages can be done to determine not only characteristics of the populations in terms of morphometry, mitosis, migration, and nucleation state, but also to compare the two passage points as an additional metric of how the cells drift and adapt to culture conditions over time.

### Co-Culture of LIP and Select Cardiac Stem Cell Types.

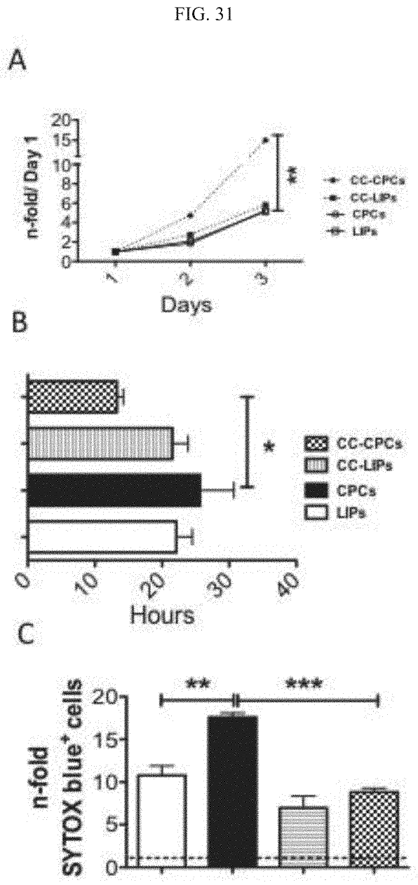

Co-culture of LIP with CPCs has marked effects upon CPC proliferation and survival in response to apoptotic stress, see FIG. 31A-C, but thorough understanding of LIPs or Lin28− CICs impact upon other cell types remain unexplored. Promising findings using combinatorial cell therapy⁴⁴ prompt determination of enhanced biological properties of CPC, MSC, and EPC when combined with LIP. Our previously published stem cell co-culture approach with neonatal rat cardiomyocytes⁸⁰ can readily be adapted with a comparable approach for assessment of LIP-mediated enhancement of widely known cardiac-derived stem cell types from human failing heart LVAD samples previously published by our group.⁶²

FIG. 31A-C graphically illustrate data showing that murine LIP co-culture improves proliferation and survival of murine CPCs. FIG. 31A graphically illustrate data showing the proliferation of LIP and mCPC in single culture and co-culture represented as a fold change relative to day of plating. FIG. 31B graphically illustrate data showing cell doubling time in hours (n=6). FIG. 31C graphically illustrate data of a cell death assay of mLIPS and mCPCs in single culture and co-culture after treatment with 80 μM H₂O₂ represented as a fold change relative to cells not subjected to H₂O₂ (n=3). CC-CPCs=CPC phenotype measured after LIP co-culture; CC-LIPs=LIP phenotype measured after CPC co-culture. CPC=CPC alone; LIP=LIP alone. *P<0.05, **P<0.01, ***P<0.001

### Functional Adaptation of LIPs Depends Upon and is Regulated by the Lin28/Let-7 Signaling Axis

The ultimate goal of understanding cardiac cell populations is to determine the optimal cell type for blunting of pathologic damage, promotion of beneficial remodeling, and ultimately enhancement of myocardial regeneration toward restoration of contractile function. Early results of LIP injection into infarcted mouse myocardium demonstrate remarkable persistence of tagged LIP cells for at least 2 months following delivery (data not shown). These preliminary findings were performed in the syngeneic context of mouse LIPs into mouse hearts.

### LIP Phenotypic and Functional Properties are Antagonized by Let-7 Expression.

Ample published literature established the antagonism of Lin28 by let-7^{9, 21, 27} as well as a critical role for let-7 in cardiogenic commitment.^{12, 81, 82} Lin28/let-7 signaling axis operation in LIPs can be confirmed through use of commercially available let-7 mimics (e.g., from Thermo-Fisher or Active Motif) with comparably treated LIPs modified with scrambled miR reagent serving as the control group. Treatments can be performed in both murine and human LIPs derived from young subjects (6-8 weeks for mice; pediatric samples from humans) to enrich for higher level Lin28 expression with empirical optimization of transfection protocols recommended by the manufacturer. Lin28 expression can be assessed by quantitative immunoblot and PCR, as well as functional activity of let-7 mimics confirmed by assays for let-7 targets (Qiagen/SABiosciences RT² Profiler™ PCR Array profiles the expression of 84 has-let-7a-5p target genes). These basic studies establish the principle of let-7 suppressing Lin28 expression in LIPs.

### Myocardial Repair Mediated by Aged Human LIPs is Enhanced by Let-7 Inhibition.

Cardioprotection mediated by let-7 inhibition has been documented in multiple publications,^{13, 15, 16, 20} however the cellular basis for diminished injury remains obscure and has been ascribed to both cardiomyocyte and CIC origins because inhibition was administered globally. Lin28 is significantly diminished in aged LIPs, so it is reasonable to posit that heightened Lin28 signaling would enhance functional capacity of aged LIPs. Implementation of autologous LIP cell therapy would require used of aged patient CPCs that would benefit from “rejuvenation” by augmentation of Lin28 activity. Therefore, in one embodiment, aged human LIPs are modified via let-7 inhibition to supplement Lin28 expression and activity.

In alternative embodiments, let-7 activity is blocked by screening of commercially available let-7 anti-miR reagents (e.g., from Thermo-Fisher or Active Motif) with empirical optimization of transfection protocols recommended by the manufacturer. Functional activity of let-7 anti-miRs can be confirmed by assays for let-7 targets (e.g., from Qiagen/SABiosciences RT² Profiler™ PCR Array profiles the expression of 84 has-let-7a-5p target genes). Lin28 expression can be assessed in at least six individual human LIP isolates from various patients to determine induction level as well as reproducibility by quantitative immunoblot as well as qPCR analyses.

Selected modified LIP with demonstrable increases in functional competency can be delivered to NOD-SCID mice coincident with infarction challenge in a protocol comparable to that previously reported by our group.⁵¹ Comparably treated LIPs modified with scrambled miR reagent can serve as the control group. Subsequent (follow-up) analyses of infarcted cell-therapy treated mice can involve typical assessments of echocardiography, immunohistochemistry, hemodynamics, molecular biology, and biochemistry in a longitudinal study following up at 2, 4, and 8 weeks potentially continuing up to months after treatment if persistent beneficial outcomes are evident as we have previously reported using modified CPCs.^{49, 51, 80}

The upregulation of let-7 can inhibit Lin28 expression and activity, impairing LIP biological and functional activities assessed by multiple criteria relative to controls. Aged LIPs with depressed Lin28 expression and activity can show improvement of biological and functional measures following delivery of let-7 anti-miR, where in alternative embodiments methods as provided herein further comprise co-administration of or delivery of let-7 anti-miR, or equivalents. LIPs exhibiting higher functional competency can lead to improved structural and functional recovery when used as cell therapeutics for treatment of infarction injury relative to control LIPs with lower functional competency.

### REFERENCES

1. Ellison G M, et al., Cardiac stem and progenitor cell identification: different markers for the same cell? Front Biosci (Schol Ed). 2010; 2:641-52.
2. Keith M C and Bolli R. “String theory” of c-kit(pos) cardiac cells: a new paradigm regarding the nature of these cells that may reconcile apparently discrepant results. Circ Res. 2015; 116:1216-30.
3. Shyh-Chang N and Daley G Q. Lin28: primal regulator of growth and metabolism in stem cells. Cell Stem Cell. 2013; 12:395-406.
4. Moss E G and Tang L. Conservation of the heterochronic regulator Lin-28, its developmental expression and microRNA complementary sites. Dev Biol. 2003; 258:432-42.
5. Zhang J, et al., LIN28 Regulates Stem Cell Metabolism and Conversion to Primed Pluripotency. Cell Stem Cell. 2016; 19:66-80.
6. Hattori A, Buac K and Ito T. Regulation of Stem Cell Self-Renewal and Oncogenesis by RNA-Binding Proteins. Adv Exp MedBiol. 2016; 907:153-88.
7. Piskounova E, Polytarchou C, Thornton J E, LaPierre R J, Pothoulakis C, Hagan J P, Iliopoulos D and Gregory R I. Lin28A and Lin28B inhibit let-7 microRNA biogenesis by distinct mechanisms. Cell. 2011; 147:1066-79.
8. Rehfeld F, Rohde A M, Nguyen D T and Wulczyn F G. Lin28 and let-7: ancient milestones on the road from pluripotency to neurogenesis. Cell Tissue Res. 2015; 359:145-60.
9. Thornton J E and Gregory R I. How does Lin28 let-7 control development and disease? Trends Cell Biol. 2012; 22:474-82.
10. Tsialikas J and Romer-Seibert J. LIN28: roles and regulation in development and beyond. Development. 2015; 142:2397-404.
11. Viswanathan S R and Daley G Q. Lin28: A microRNA regulator with a macro role. Cell. 2010; 140:445-9.
12. Bao M H, Feng X, Zhang Y W, Lou X Y, Cheng Y and Zhou H H. Let-7 in cardiovascular diseases, heart development and cardiovascular differentiation from stem cells. Int J Mol Sci. 2013; 14:23086-102.
13. Li J, Ren Y, Shi E, Tan Z, Xiong J, Yan L and Jiang X. Inhibition of the Let-7 Family MicroRNAs Induces Cardioprotection Against Ischemia-Reperfusion Injury in Diabetic Rats. Ann Thorac Surg. 2016; 102:829-35.
14. Satoh M, Minami Y, Takahashi Y, Tabuchi T and Nakamura M. A cellular microRNA, let-7i, is a novel biomarker for clinical outcome in patients with dilated cardiomyopathy. J Card Fail. 2011; 17:923-9.
15. Seeger T, Xu Q F, Muhly-Reinholz M, Fischer A, Kremp E M, Zeiher A M and Dimmeler S. Inhibition of let-7 augments the recruitment of epicardial cells and improves cardiac function after myocardial infarction. J Mol Cell Cardiol. 2016; 94:145-52.
16. Tolonen A M, Magga J, Szabo Z, Viitala P, Gao E, Moilanen A M, Ohukainen P, Vainio L, Koch W J, Kerkela R, Ruskoaho H and Serpi R. Inhibition of Let-7 microRNA attenuates myocardial remodeling and improves cardiac function postinfarction in mice. Pharmacol Res Perspect. 2014; 2:e00056.
17. Aguirre A, et al., In vivo activation of a conserved microRNA program induces mammalian heart regeneration. Cell Stem Cell. 2014; 15:589-604.
18. Du Y, et al., Let-7a regulates expression of beta1-adrenoceptors and forms a negative feedback circuit with the beta1-adrenoceptor signaling pathway in chronic ischemic heart failure. Oncotarget. 2017; 8:8752-8764.
19. Joshi S, Wei J and Bishopric N H. A cardiac myocyte-restricted Lin28/let-7 regulatory axis promotes hypoxia-mediated apoptosis by inducing the AKT signaling suppressor PIK3IP 1. Biochim Biophys Acta. 2016; 1862:240-51.
20. Gray C, Li M, Patel R, Reynolds C M and Vickers M H. Let-7 miRNA profiles are associated with the reversal of left ventricular hypertrophy and hypertension in adult male offspring from mothers undernourished during pregnancy after preweaning growth hormone treatment. Endocrinology. 2014; 155:4808-17.
21. Shi F, Yu W and Wang X. Bistable switch in let-7 miRNA biogenesis pathway involving Lin28. Int J Mol Sci. 2014; 15:19119-33.
22. Jun-Hao E T, Gupta R R and Shyh-Chang N. Lin28 and let-7 in the Metabolic Physiology of Aging. Trends Endocrinol Metab. 2016; 27:132-41.
23. Vogt E J, Meglicki M, Hartung K I, Borsuk E and Behr R. Importance of the pluripotency factor LIN28 in the mammalian nucleolus during early embryonic development. Development. 2012; 139:4514-23.
24. Zhou J, Ng S B and Chng W J. LIN28/LIN28B: an emerging oncogenic driver in cancer stem cells. Int J Biochem Cell Biol. 2013; 45:973-8.
25. Shinoda G, et al., Fetal deficiency of lin28 programs life-long aberrations in growth and glucose metabolism. Stem Cells. 2013; 31:1563-73.
26. Shyh-Chang N, et al., Lin28 enhances tissue repair by reprogramming cellular metabolism. Cell. 2013; 155:778-92.
27. Zhu H, et al., The Lin28/let-7 axis regulates glucose metabolism. Cell. 2011; 147:81-94.
28. Burkhalter M D, Morita Y and Rudolph K L. Lin28a—boost your energy for youthful regeneration. EMBO J. 2014; 33:5-6.
29. Reddien P W. Lin28: time for tissue repair. Cell. 2013; 155:738-9.
30. Wrighton K H. Metabolism: Young again with Lin28. Nat Rev Mol Cell Biol. 2014; 15:4.
31. Corre C, Shinoda G, Zhu H, Cousminer D L, Crossman C, Bellissimo C, Goldenberg A, Daley G Q and Palmert M R. Sex-specific regulation of weight and puberty by the Lin28/let-7 axis. J Endocrinol. 2016; 228:179-91.
32. Piskounova E, et al., Determinants of microRNA processing inhibition by the developmentally regulated RNA-binding protein Lin28. J Biol Chem. 2008; 283:21310-4.
33. Viswanathan S R, et al., Lin28 promotes transformation and is associated with advanced human malignancies. Nat Genet. 2009; 41:843-8.
34. West J A, et al., A role for Lin28 in primordial germ-cell development and germ-cell malignancy. Nature. 2009; 460:909-13.
35. Tsanov K M, et al., LIN28 phosphorylation by MAPK/ERK couples signalling to the post-transcriptional control of pluripotency. Nat Cell Biol. 2017; 19:60-67.
36. Urbach A, Yermalovich A, Zhang J, Spina C S, Zhu H, Perez-Atayde A R, Shukrun R, Charlton J, Sebire N, Mifsud W, Dekel B, Pritchard-Jones K and Daley G Q. Lin28 sustains early renal progenitors and induces Wilms tumor. Genes Dev. 2014; 28:971-82.
37. Feng C, et al., Lin28 regulates HER2 and promotes malignancy through multiple mechanisms. Cell Cycle. 2012; 11:2486-94.
38. Tu H C, et al., LIN28 cooperates with WNT signaling to drive invasive intestinal and colorectal adenocarcinoma in mice and humans. Genes Dev. 2015; 29:1074-86.
39. Chang T C, Zeitels L R, Hwang H W, Chivukula R R, Wentzel E A, Dews M, Jung J, Gao P, Dang C V, Beer M A, Thomas-Tikhonenko A and Mendell J T. Lin-28B transactivation is necessary for Myc-mediated let-7 repression and proliferation. Proc Natl Acad Sci USA. 2009; 106:3384-9.
40. Yu J, et al., Induced pluripotent stem cell lines derived from human somatic cells. Science. 2007; 318:1917-20.
41. Keane M and de Magalhaes J P. MYCN/LIN28B/Let-7/HMGA2 pathway implicated by meta-analysis of GWAS in suppression of post-natal proliferation thereby potentially contributing to aging. Mech Ageing Dev. 2013; 134:346-8.
42. Silveira-Neto A P, et al., Absence of functional LIN28B mutations in a large cohort of patients with idiopathic central precocious puberty. Horm Res Paediatr. 2012; 78:144-50.
43. Toledano H. The role of the heterochronic microRNA let-7 in the progression of aging. Exp Gerontol. 2013; 48:667-70.
44. Williams A R, et al., Enhanced effect of combining human cardiac stem cells and bone marrow mesenchymal stem cells to reduce infarct size and to restore cardiac function after myocardial infarction. Circulation. 2013; 127:213-23.
45. Cheng K, et al., Relative roles of CD90 and c-kit to the regenerative efficacy of cardiosphere-derived cells in humans and in a mouse model of myocardial infarction. J Am Heart Assoc. 2014; 3:e001260.
46. van Berlo J H and Molkentin J D. Most of the Dust Has Settled: cKit+ Progenitor Cells Are an Irrelevant Source of Cardiac Myocytes In Vivo. Circ Res. 2016; 118:17-9.
47. Vicinanza C, et al., Adult cardiac stem cells are multipotent and robustly myogenic: c-kit expression is necessary but not sufficient for their identification. Cell Death Differ. 2017; 24:2101-2116.
48. Aquila I, et al., The use and abuse of Cre/Lox recombination to identify adult cardiomyocyte renewal rate and origin. Pharmacol Res. 2017.
49. Fischer K M, et al., Enhancement of myocardial regeneration through genetic engineering of cardiac progenitor cells expressing Pim-1 kinase. Circulation. 2009; 120:2077-87.
50. Fransioli J, et al., Evolution of the c-kit-positive cell response to pathological challenge in the myocardium. Stem Cells. 2008; 26:1315-24.
51. Mohsin S, et al., Human cardiac progenitor cells engineered with Pim-I kinase enhance myocardial repair. J Am Coll Cardiol. 2012; 60:1278-87.
52. Shi H, et al., Hiding inside? Intracellular expression of non-glycosylated c-kit protein in cardiac progenitor cells. Stem CellRes. 2016; 16:795-806.
53. Castaldi A, Dodia R M, Orogo A M, Zambrano C M, Najor R H, Gustafsson A B, Heller Brown J and Purcell N H. Decline in cellular function of aged mouse c-kit(+) cardiac progenitor cells. J Physiol. 2017; 595:6249-6262.
54. Li T S, et al., Direct comparison of different stem cell types and subpopulations reveals superior paracrine potency and myocardial repair efficacy with cardiosphere-derived cells. J Am Coll Cardiol. 2012; 59:942-53.
55. Ibrahim A G, Cheng K and Marban E. Exosomes as critical agents of cardiac regeneration triggered by cell therapy. Stem Cell Reports. 2014; 2:606-19.
56. Khan M and Kishore R. Stem Cell Exosomes: Cell-FreeTherapy for Organ Repair. Methods Mol Biol. 2017; 1553:315-321.
57. Kishore R and Khan M. More Than Tiny Sacks: Stem Cell Exosomes as Cell-Free Modality for Cardiac Repair. Circ Res. 2016; 118:330-43.
58. Kishore R and Khan M. Cardiac cell-derived exosomes: changing face of regenerative biology. Eur Heart J. 2017; 38:212-215.
59. Newman R H and Zhang J. Fucci: street lights on the road to mitosis. Chem Biol. 2008; 15:97-8.
60. Singh A M, Trost R, Boward B and Dalton S. Utilizing FUCCI reporters to understand pluripotent stem cell biology. Methods. 2016; 101:4-10.
61. Zielke N and Edgar B A. FUCCI sensors: powerful new tools for analysis of cell proliferation. Wiley Interdiscip Rev Dev Biol. 2015; 4:469-87.
62. Monsanto M M, et al., Concurrent Isolation of 3 Distinct Cardiac Stem Cell Populations From a Single Human Heart Biopsy. Circ Res. 2017; 121:113-124.
63. Li Y, Lin B and Yang L. Comparative transcriptomic analysis of multiple cardiovascular fates from embryonic stem cells predicts novel regulators in human cardiogenesis. Sci Rep. 2015; 5:9758.
64. Mohsin S, et al., Rejuvenation of human cardiac progenitor cells with Pim-1 kinase. Circ Res. 2013; 113:1169-79.
65. Kulandavelu S, et al., Piml Kinase Overexpression Enhances ckit+ Cardiac Stem Cell Cardiac Repair Following Myocardial Infarction in Swine. J Am Coll Cardiol. 2016; 68:2454-2464.
66. Muraski J A, et al., Pim-1 regulates cardiomyocyte survival downstream of Akt. Nat Med. 2007; 13:1467-75.
67. Siddiqi S, et al., Myocardial induction of nucleostemin in response to postnatal growth and pathological challenge. Circ Res. 2008; 103:89-97.
68. Ivey M J, et al., Resident fibroblast expansion during cardiac growth and remodeling. J Mol Cell Cardiol. 2017; 114:161-174.
69. Ivey M J and Tallquist M D. Defining the Cardiac Fibroblast. Circ J. 2016; 80:2269-2276.
70. Tallquist M D and Molkentin J D. Redefining the identity of cardiac fibroblasts. Nat Rev Cardiol. 2017; 14:484-491.
71. Shinoda G, et al., Lin28a regulates germ cell pool size and fertility. Stem Cells. 2013; 31:1001-9.
72. Hariharan N, et al., Nucleostemin rejuvenates cardiac progenitor cells and antagonizes myocardial aging. J Am Coll Cardiol. 2015; 65:133-47.
73. Bailey B, Fransioli J, Gude N A, Alvarez R, Jr., Zhang X, Gustafsson A B and Sussman M A. Sca-1 knockout impairs myocardial and cardiac progenitor cell function. Circ Res. 2012; 111:750-60.
74. Muraski J A, et al., Pim-1 kinase antagonizes aspects of myocardial hypertrophy and compensation to pathological pressure overload. Proc Natl Acad Sci USA. 2008; 105:13889-94.
75. Sussman M A, et al., Myofibril degeneration caused by tropomodulin overexpression leads to dilated cardiomyopathy in juvenile mice. J Clin Invest. 1998; 101:51-61.
76. Sussman M A, et al., Pathogenesis of dilated cardiomyopathy: molecular, structural, and population analyses in tropomodulin-overexpressing transgenic mice. Am J Pathol. 1999; 155:2101-13.
77. Tsujita Y, Muraski J, Shiraishi I, Kato T, Kajstura J, Anversa P and Sussman M A. Nuclear targeting of Akt antagonizes aspects of cardiomyocyte hypertrophy. Proc Natl Acad Sci USA. 2006; 103:11946-51.
78. Welch S, et al., Cardiac-specific IGF-1 expression attenuates dilated cardiomyopathy in tropomodulin-overexpressing transgenic mice. Circ Res. 2002; 90:641-8.
79. Zheng G X, et al., Massively parallel digital transcriptional profiling of single cells. Nat Commun. 2017; 8:14049.
80. Quijada P, et al., Cardiac Stem Cell Hybrids Enhance Myocardial Repair. Circ Res. 2015; 117:695-706.
81. Hashemzadeh M. Role of micro RNAs in stem cells, cardiac differentiation and cardiovascular diseases. Gene Reports. 2017; 8:11-16.
82. Kuppusamy K T, et al., Let-7 family of microRNA is required for maturation and adult-like metabolism in stem cell-derived cardiomyocytes. Proc Natl Acad Sci USA. 2015; 112:E2785-94.

Although the invention has been described in the context of certain embodiments, it is intended that the patent will not be limited to those embodiments; rather, the scope of this patent shall encompass the full lawful scope of the appended claims, and lawful equivalents thereof.

1: A method for identifying or isolating a cardiac stem cell, an adult cardiac stem cell or a cardiac progenitor cell, comprising: identifying a cell or cells expressing:
(i) a Lin28 polypeptide, or a Lin-28 homolog A, a protein that in humans is encoded by the LIN28 gene, or a Lin28+ cell, or
(ii) a transcript (mRNA) encoding the Lin28 polypeptide.

2. A macrocellular structure, a cardiocluster of cells, or an artificially configured plurality of cells, comprising:
(a) (i) a core region or cluster comprising a plurality of Lin28+ cardiac stem cells or Lin28+ cardiac progenitor cells, or cells isolated using a method of claim 1, or a combination thereof; and
(ii) a second region or a peripheral region positioned: at least partially surrounding the outer surface of the core region or cluster, or at least partially around the core region or cluster, comprising a plurality of second cardiac stem cells, wherein the first cardiac stem cells and the second cardiac stem cells are different, and the second cardiac progenitor cells are selected from the group consisting of:
a plurality of mesenchymal stem cells or mesenchymal progenitor cells,
a plurality of endothelial progenitor cells or endothelial stem cells, and
a combination thereof; or
(b) (i) a core region or cluster comprising: a plurality of first cardiac stem cells and second cardiac stem cells, wherein the first cardiac stem cells and the second cardiac stem cells are different, and the first cardiac progenitor cells are selected from the group consisting of:
a plurality of mesenchymal stem cells or mesenchymal progenitor cells,
a plurality of endothelial progenitor cells or endothelial stem cells, and
a combination thereof; and
(ii) a second region or a peripheral region positioned: at least partially surrounding the outer surface of the core region or cluster, or at least partially around the core region or cluster, comprising a plurality of second cardiac stem cells comprising: a plurality of Lin28+ cardiac stem cells or Lin28+ cardiac progenitor cells, cells isolated using a method of claim 1, or a combination thereof.

3: A macrocellular structure, cardiocluster of cells, or the artificially configured plurality of cells comprising a first, a second and a third plurality of stem cells or progenitor cells, wherein the first, second and third cardiac stem cells or progenitor cells are different cell types or comprises a different set of cells,
and at least one of the first, second and third cardiac stem cells or progenitor cells comprise:
(i) a plurality of cardiac progenitor cells or cardiac stem cells,
a plurality of mesenchymal stem cells or mesenchymal progenitor cells,
a plurality of endothelial progenitor cells or endothelial stem cells, or
a combination thereof, and
(ii) a plurality of Lin28+ cardiac stem cells or Lin28+ cardiac progenitor cells, or cells isolated using a method of claim 1.

4: The macrocellular structure, cardiocluster of cells, or the artificially configured plurality of cells of claim 2, wherein
(a) the first, second, and third stem cells are of human origin or are of non-human (animal) origin, or, the core region or cluster, or
(b) the second or third region or peripheral region, comprises cells selected from the group consisting of: c-kit+ cardiac progenitor cells (CPCs), CD90+/CD105+ mesenchymal stem cells (MSCs) CD133+ endothelial progenitor cells (EPCs), and a combination thereof.

5: A product of manufacture comprising:
(a) a cell or a plurality of Lin28+ cardiac stem cells or Lin28+ cardiac progenitor cells, or a cell or a plurality of cells isolated using a method of claim 1,
(b) a macrocellular structure, a cardiocluster of cells or an artificially configured plurality of cells as set forth in claim 2,
(c) any combination of (a) and (b).

6: A method for:
inducing or accelerating cardiogenesis in the mammalian heart,
initiating, inducing or accelerating tissue repair or tissue regeneration,
initiating, inducing or accelerating a cardiac muscle repair or tissue regeneration, a cardiac vasculature repair or tissue regeneration or a cardiac connective tissue repair or tissue regeneration,
comprising:
(a) providing:
a cell or a plurality of Lin28+ cardiac stem cells, or a cell or a plurality of Lin28+ cardiac progenitor cells, or
a cell or plurality of cells isolated using a method of claim 1; and
(b) introducing into, onto or approximate to the mammalian heart, or cardiac or heart tissue, or heart muscle, or cardiac vasculature or connective tissue: the plurality of Lin28+ cardiac stem cells or Lin28+ cardiac progenitor cells, or the plurality of cells isolated using a method of claim 1,
thereby inducing or accelerating cardiogenesis in the mammalian heart, or for repairing or regenerating the tissue, or the cardiac tissue, or the cardiac muscle, cardiac vasculature or cardiac connective tissue.

7: The method of claim 6, wherein heart has an injury or dysfunction and the method is effective to treat the injury or dysfunction.

8: The method of claim 7, wherein injury or dysfunction: is an ischemic injury or a heart failure, or results from myocardial infarction (MI).

9: A method for treating or ameliorating a heart injury, an injury subsequent to or following from 1 minute to 12 hours after, or immediately after a myocardial infarction (MI), a congenital or genetic heart defect, or a heart dysfunction, comprising:
(a) providing:
a cell or a plurality of Lin28+ cardiac stem cells, or a cell or a plurality of Lin28+ cardiac progenitor cells, or
a cell or plurality of cells isolated using a method of claim 1; and
(b) introducing into, onto or approximate to (close to) a mammalian heart, or administering to or applying to an individual in need thereof, the plurality of Lin28+ cardiac stem cells or Lin28+ cardiac progenitor cells, or the plurality of cells isolated using a method of claim 1,
thereby treating or ameliorating the heart injury, injury subsequent to or following from 1 minute to 12 hours after, or immediately after a myocardial infarction (MI), congenital or genetic heart defect, or heart dysfunction.

10: A method for treating or ameliorating a heart injury, an injury subsequent to or following from 1 minute to 12 hours after, or immediately after a myocardial infarction (MI), a congenital or genetic heart defect, or a heart dysfunction, comprising:
(a) providing: a product of manufacture of claim 5; and
(b) introducing into, onto or approximate to a mammalian heart, or administering to or applying to an individual in need thereof, the product of manufacture of (a),
thereby treating or ameliorating the heart injury, injury subsequent to (or following, or immediately after) a myocardial infarction (MI), congenital or genetic heart defect, or heart dysfunction.

11-12. (canceled)

13: The method of claim 1, further comprising a step (b) comprising isolating the identified Lin28+ cells.

14: The method of claim 13, wherein the cells that are Lin28+ are isolated using polypeptides or compositions that can specifically or non-specifically bind Lin28, or substantially bind Lin28, and optionally the polypeptides are Lin28-binding antibodies or Lin28-binding fragments thereof.

15: The method of claim 1, wherein the cells expressing the transcripts (mRNA) encoding the Lin28 polypeptide are identified using fluorescence in situ hybridization (FISH) or polymerase chain reaction (PCR),
and optionally the PCR comprises a real-time PCR or a quantitative polymerase chain reaction (qPCR)), or an in situ hybridization (ISH).

16: The method of claim 1, wherein the cells are human or non-human or animal cells.

17: The method of claim 3, wherein the plurality of third stem cell are positioned or configured: at least partially surrounding the outer surface of the second region or the core region or cluster, or at least partially around the second region or the core region or cluster,
and optionally the plurality of third stem cells or progenitor cells are positioned or configured in the core region or cluster,
and optionally the plurality of third stem cell are positioned or configured: at least partially surrounding the outer surface of the second region or peripheral region or the core region or cluster, or at least partially around the second region or peripheral region or the core region or cluster, and are also positioned or configured in the core region or cluster,
and optionally the plurality of third stem cells or progenitor cells are of non-cardiac origin, and optionally the third stem cells are mesenchymal stem cells of non-cardiac origin.

18: The product of manufacture of claim 5, wherein the product of manufacture comprises or is manufactured as a drug delivery device, an implant, a catheter, a cartridge, an ampoule, a stent, or a medical device.

19: The method of claim 6, wherein the initiating, inducing or accelerating tissue repair or tissue regeneration comprises initiating, inducing or accelerating a cardiac or heart tissue repair or heart tissue regeneration.
